(19) United States
(12) Patent Application Publication
(10) Pub. No.: US 2011/0105986 A1
(43) Pub. Date: May 5, 2011

(54) UVEOSCLERAL DRAINAGE DEVICE

(76) Inventors: Ben Bronstein, (US); Nicholas Fish Warner, (US); Sue Warner, (US); M. Bruce Shields, Hamden, CT (US); Juan F. Battle, Santo Domingo, CA (US)

(21) Appl. No.: 12/887,488

(22) Filed: Sep. 21, 2010

Related U.S. Application Data
(60) Provisional application No. 61/244,113, filed on Sep. 21, 2009.

(51) Int. Cl. A61M 5/00

(52) U.S. Cl. 604/8

(57) ABSTRACT

An ophthalmic shunt implantable in an eye having an elongate body and a branched conduit for conducting aqueous humor from an anterior chamber of the eye to the suprachoroidal space of the eye and the subconjunctival space, and a plate extending from an upper surface of the elongate body.

### CROSS-REFERENCE TO RELATED APPLICATIONS

This application claims priority from U.S. Provisional Application No. 61/244,113 entitled “Uveoscleral Drainage Device” filed Sep. 21, 2009, the contents of which are hereby incorporated by reference in their entirety.

### GOVERNMENT INTERESTS

Not applicable

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

Not applicable

### SUMMARY

Various embodiments of the invention described herein are directed to an ophthalmic shunt implantable in an eye including an elongate body having a forward end, a spaced back end, an upper surface, and a lower surface, an insertion head extending from the forward end of the elongate body and being continuous with the elongate body, the insertion head defining a shearing edge constructed and arranged for cutting eye tissue, a conduit having a first end defined on the insertion head and a first branch extending through the elongate body from the forward end to the back end of the elongate body and a second branch extending through the elongate body to the upper surface of the elongate body, a connector extending from the upper surface of the elongate body, the connector encompassing and lengthening the second branch, wherein the second branch forms a lumen within the connector, and a plate having an upper and a lower surface, the lower surface of the plate extending from the connector opposite the elongate body and the connector creating a space between the upper surface of the elongate body and the lower surface of the plate.

Various other embodiments are directed to an ophthalmic shunt implantable in an eye, including: an elongate body having a forward end, a spaced back end, an upper surface, and a lower surface; an insertion head extending from the forward end of the elongate body and being continuous with the elongate body, the insertion head defining a shearing edge constructed and arranged for cutting eye tissue, the forward end of the elongate body and the insertion head further defining a shoulder surface; a conduit having a first end defined on the insertion head and a first branch extending through the elongate body from the forward end to the back end of the elongate body and a second branch extending through the elongate body to the upper surface of the elongate body; a connector extending from the upper surface of the elongate body, the connector encompassing and lengthening the second branch, wherein the second branch forms a lumen within the connector; and a plate having an upper and a lower surface, the lower surface of the plate extending from the connector opposite the elongate body and the connector creating a space between the upper surface of the elongate body and the lower surface of the plate.

In some embodiments, the elongate body may be configured to position at least a portion of the insertion head and the first end of the conduit through an incision formed by the shearing edge of the insertion head and into fluid communication with the anterior chamber of the eye. In other embodiments, the elongate body may have an arcuate shape along at least a portion of its length that is adapted to extend along the curvature of the sclera, and in still other embodiments, the elongate body may have a substantially fusiform cross-sectional shape.

In some embodiments, the plate may extend beyond at least one edge of the elongate body, and in other embodiments, the plate may have a shape selected from polygonal, rounded polygonal, circular, oval, and elliptical. In still other embodiments, the plate may be contoured to cover at least a portion of the sclera. In some embodiments, at least one axis of the plate may have a diameter or width of greater than about 2 mm, and in other embodiments, at least one axis of the plate may have a diameter or width of from about 3 mm to about 9 mm. In certain embodiments, at least one axis of the plate has a diameter or width of about 6 mm. In some embodiments, the upper surface of the plate may be convex, and in certain embodiments, the convex upper surface of the plate may have a curvature that is substantially similar to adjacent sclera when the shunt is implanted. In other embodiments, the upper surface of the elongate body and the lower surface of the plate may be arranged and spaced to receive sclera of an eye. In still other embodiments, the lower surface of the plate may be substantially flat. In further embodiments, the lower surface of the plate is concave, and in particular embodiments, the concave lower surface of the plate may have a curvature that is steeper than adjacent sclera when the shunt is implanted such that a convex space is created between the sclera and the lower surface of the plate when the shunt is implanted. In some embodiments, at least a portion of the lower surface of the plate may be textured, and in various such embodiments, the texture of the textured lower surface may be selected from corrugations, fingers, bumps, concentric circles or portions of concentric circles and combinations thereof. In some embodiments, the upper surface of the plate may be substantially co-planar with the lower surface of the elongate body. In other embodiments, the upper surface of the plate is convex and the lower surface of plate is concave, and in certain embodiments, the curvature of the convex upper surface and the curvature of the concave lower surface is substantially the same.

In some embodiments, the connector may have a height of from about 0.5 mm to about 0.8 mm from the upper surface of the elongate body to the lower surface of the plate, and in other embodiments, the connector may have a height of about 0.6 mm from the upper surface of the elongate body to the lower surface of the plate. In further embodiments, a joint between the connector and the plate may be at a midpoint of the plate. In some embodiments, a joint between the connector and the plate may be offset from a midpoint of the plate, and in particular embodiments, the joint between the connector and the plate is offset toward an anterior portion of the plate. In still other embodiments, the joint between the connector and the plate may be positioned such than an outer surface of the connector and an outer edge of the plate are separated by about 2 mm or more, and in certain embodiments, the outer edge of the plate may be defined on an anterior portion of the plate. In some embodiments, the connector may be separated from the back end of the elongate body by about 1 mm or more. In certain embodiments, at least a portion of the lumen of the connector may include a flow regulator. In such embodiments, the flow regulator may be selected from a valve, a membrane, a porous material, a flap or a combination thereof, and in particular embodiments, the membrane or the porous material can be at least partially removed by laser. In other embodiments, the membrane or the porous material is biodegradable or non-biodegradable. In some embodiments, a second end of the second branch is defined on the upper surface of the plate. In such embodiments, the upper surface of the plate further comprises a flow regulator position to regulate the flow of liquid through the second end of the conduit, and in certain embodiments, the flow regulator may be selected from, a valve, a membrane, a porous material, a flap or a combination thereof. in some embodiments, the membrane or the porous material can be at least partially removed by laser, and in other embodiments, the membrane or the porous material may be biodegradable or non-biodegradable. In some embodiments, a second end of the second branch may be defined on a portion of the connector. In such embodiments, the second end of the second branch may be positioned below the lower surface of the plate, and in some embodiments, the second end of the second branch may include one or more openings in the connector perpendicular to the lumen. In other embodiments, the upper surface of the plate may be continuous such that no opening for the second branch is defined on the upper surface of the plate. In some embodiments, an aperture to the second branch may be defined on an upper surface of the plate, and in certain embodiments, the aperture may include a flow regulator position to regulate the flow of liquid through the aperture. In other embodiments, the flow regulator may be selected from a valve, a membrane, a porous material, a flap or a combination thereof, and in particular embodiments, the membrane or the porous material can be at least partially removed by laser.

In various embodiments, the shunt may be prepared from a material selected from biocompatible metals, gold, platinum, nickel, molybdenum, titanium, biocompatible metal alloys, biocompatible polymers, silicone and combinations thereof. In some embodiments, the elongate body may be prepared from a rigid or semi-rigid material, and in other embodiments, the plate may be prepared from a flexible material. In some embodiments, the plate may be prepared from silicone, and in other embodiments, the plate may be prepared from a flexible biocompatible polymer. In some embodiments, the connector may be prepared from a material selected from a flexible material, a semi-rigid material, and a rigid material. In particular embodiments, the connector may further include a suture encircling the connector and positioned and arranged to obstruct flow of fluid through the connector, and in some embodiments, the suture may be selected from releasable sutures, biodegradable sutures or combinations thereof.

In particular embodiments, the shunt may further include one or more therapeutic agents. In some embodiments, the therapeutic agent may be selected from steroids, beta blockers, alpha-2 antagonists, carbonic anhydride inhibitors, prostaglandin analogues, anti-fibrotic agents, anti-inflammatory agents, and antimicrobial agents. In some embodiments, the one or more therapeutic agents may be contained within the conduit, the first branch, the second branch, or combinations thereof, and in other embodiments, the one or more therapeutic agents are coated on outer or inner surfaces of the elongate body, coated on outer or inner surfaces of the insertion head, coated on outer or inner surfaces of the connector, coated on outer or inner surfaces of the plate, or combinations thereof.

Some embodiments of the invention are directed to a method for treating glaucoma in an eye including the steps of inserting at least a portion of a first end of a biocompatible ophthalmic shunt through the sclera and suprachoroidal space into the anterior chamber of an eye such that at least a portion of the first end is in fluid communication with the anterior chamber of the eye; positioning a second portion of the shunt into a suprachoroidal space of the eye such that at least a portion the second portion of the shunt is in fluid communication with the suprachoroidal space; and positioning a third portion of the shunt into the subconjunctival space of the eye such that at least a portion of the third portion of the shunt is in communication with the subconjunctival space.

In some embodiments, the first end, the second portion, and the third portion are connected by a branched conduit, and in other embodiments, flow of fluid through the third portion of the shunt may be at least partially obstructed when the third portion is initially positioned. In some embodiments, the method may further include removing the obstruction when flow of fluid through the second portion becomes blocked and/or pressure within the anterior chamber of the eye is insufficiently reduced to effect treatment, and in other embodiments, the method may further include applying a suture to the third portion of the shunt to obstruct flow of fluid through the third portion of the shunt. In such embodiments, the suture may be selected from releasable sutures and biodegradable sutures, and in such embodiments, the method may further include releasing the suture when flow of fluid through the second portion becomes blocked or pressure within the anterior chamber of the eye is insufficiently reduced to effect treatment. In some embodiments, the shunt may include a flow regulator selected from a membrane, a porous material, or a combination thereof, and the method may further include removing at least a portion of the membrane, porous material, or a combination thereof when flow of fluid through the second portion becomes blocked and/or pressure within the anterior chamber of the eye is insufficiently reduced to effect treatment. In such embodiments, the method may further include removing at least a portion of the membrane, porous material, or a combination thereof by applying a laser to the membrane, porous material or combination thereof. In other embodiments, the shunt may include a flow regulator selected from a valve, a flap or a combination thereof, and the method may further include opening the valve, flap, or a combination thereof when flow of fluid through the second portion becomes blocked and/or pressure within the anterior chamber of the eye is insufficiently reduced to effect treatment.

Other embodiments of the invention are directed to a method for treating glaucoma in an eye including the steps of providing a biocompatible ophthalmic shunt including: an elongate body having a forward end, a spaced back end, an upper surface, and a lower surface; an insertion head extending from the forward end of the elongate body and being continuous with the elongate body, the insertion head defining a shearing edge constructed and arranged for cutting eye tissue; a conduit having a first end defined on the insertion head and a first branch extending through the elongate body from the forward end to the back end of the elongate body and a second branch extending through the elongate body to the upper surface of the elongate body; a connector extending from the upper surface of the elongate body, the connector encompassing and lengthening the second branch, wherein the second branch forms a lumen within the connector; a plate having an upper and a lower surface, the lower surface of the plate extending from the connector opposite the elongate body and the connector creating a space between the upper surface of the elongate body and the lower surface of the plate; inserting at least a portion of the shearing edge of the insertion head of the shunt into and through an anterior chamber angle into the anterior chamber of an eye wherein at least the first end of the conduit is in fluid communication with the anterior chamber of the eye following insertion; positioning the back end of the elongate body into a suprachoroidal space of the eye so that a second end of the conduit is in fluid communication with the suprachoroidal space; and positioning the plate such that the upper surface of the plate is exposed to the subconjunctival space of the eye.

In some embodiments, the method may include making an incision in and through the conjunctiva and the sclera at a position posterior to the limbus, and in other embodiments, the method may further include positioning the plate to cover or traverse an incision made for insertion of the shunt. In certain embodiments, the plate may be made from a flexible material and the method may further include lifting a portion of the plate to expose at least a portion of the incision, suturing the incision, and replacing the plate. In some embodiments, the method may further include delivering one or more therapeutic agents, and in such embodiments, the therapeutic agent may be delivered to a portion of the eye selected from the anterior chamber, the subconjunctival space, the suprachoroidal space, and combinations thereof.

### DESCRIPTION OF THE DRAWINGS

For a fuller understanding of the nature and advantages of the present invention, reference should be made to the following detailed description taken in connection with the accompanying drawings, in which:

FIG. 1 is an illustration of the human eye showing various structural elements.

FIG. 2A shows an embodiment of the shunt of the invention having a tubular elongate body and insertion head.

FIG. 2B shows an embodiment of the shunt of the invention having a flattened elongate body and insertion head.

FIG. 3 shows a cross-sectional view of an embodiment of the shunt of the invention.

FIG. 4A shows an embodiment of the shunt of the invention having a plate in which the distance between the connector and the outer edge of the plate is equal on all sides.

FIG. 4B shows an embodiment of the shunt of the invention having plate in which the connector is offset toward the anterior of the device.

FIG. 4 C shows an embodiment of the shunt of the invention having a plate in which the connector is offset toward the anterior of the device.

FIG. 5 shows an embodiment of the shunt of the invention having a plate that is offset toward the anterior of the device and a connector that is offset toward the posterior of the plate and illustrates the taper of the plate.

FIG. 6 shows an embodiment of the shunt of the invention having a space or gap beneath the plate to accommodate liquid exiting from openings located beneath the plate.

FIG. 7 shows an embodiment of the shunt of the invention having a shaped connector.

FIG. 8 shows the connector of an embodiment of the shunt of the invention.

FIG. 9 shows an embodiment of the shunt of the invention having a valve contained within the connector.

FIG. 10 shows an embodiment of the shunt of the invention having a porous material contained within the connector.

FIG. 11 shows an embodiment of the shunt of the invention having a membrane that encompasses an opening on an outer surface of the plate.

FIG. 12 shows an embodiment of the shunt of the invention having a tubular elongate body and insertion head.

FIG. 13 shows an embodiment of the shunt of the invention having a flattened elongate body and insertion head.

FIG. 14 shows a cross-sectional view of an embodiment of the shunt of the invention having a flattened elongate body and insertion head.

FIG. 14A shows a top view of an embodiment of the shunt of the invention having a flattened elongate body and insertion head.

FIG. 15 shows an embodiment of the shunt of the invention having longitudinal grooves and suture holes.

### DETAILED DESCRIPTION

Before the compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims.

It must be noted that, as used herein, and in the appended claims, the singular forms “a”, “an” and “the” include plural reference unless the context clearly dictates otherwise. Unless defined otherwise, all technical and scientific terms used herein have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%.

Glaucoma, a leading cause of world blindness, is a group of disorders, characterized by irreversible damage to the optic nerve, or glaucomatous optic neuropathy, in which elevated intraocular pressure is the main causative risk factor. A proven way to prevent the blindness of glaucoma is to control the intraocular pressure.

Clinical management of intraocular pressure can be achieved medically or surgically. Modern medical therapy for glaucoma began in the 1870s, with the introduction of pilocarpine and other cholinergic agonists. In the twentieth century, several compounds were introduced, such as alpha-2 agonists, beta-adrenergic antagonists, topical and systemic carbonic anhydrase inhibitors, and prostaglandins. However, glaucoma medication is not available or practical in many parts of the world, and is inadequate in many patients, despite availability. Hence the need for surgical methods to control the intraocular pressure.

Control of intraocular pressure can be achieved surgically by reducing the production of aqueous humor or by increasing its outflow. Operations to reduce production, referred to collectively as cyclodestructive surgery, destroy a portion of the ciliary body, the source of aqueous humor. Destructive elements over the years have included diathermy, cryotherapy and, most recently, laser energy. While these operations are effective in lowering the intraocular pressure and are beneficial in certain situations, they have a high complication rate, including inflammation and further reduction in visual acuity.

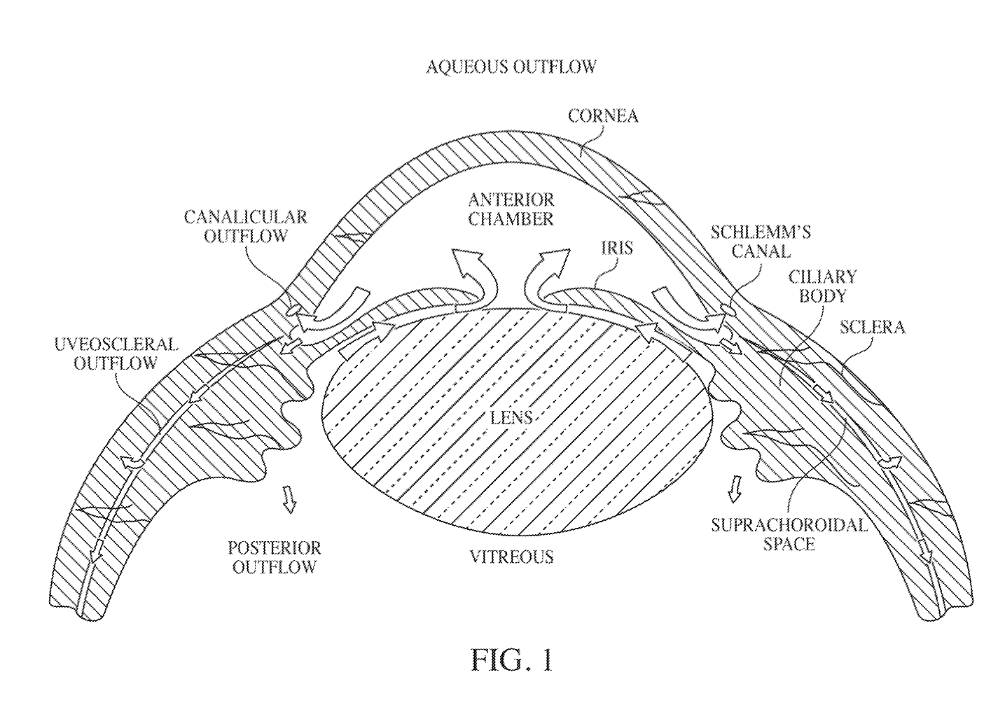

Referring to FIG. 1, after production by the ciliary body, aqueous humor leaves the eye by many routes. Some goes posteriorly through the vitreous body to the retina, while most circulates in the anterior segment of the eye, nourishing avascular structures such as the lens and cornea, before outflow by two main routes: canalicular or uveoscleral.

The canalicular route, also referred to as the trabecular or conventional route, is the main mechanism of outflow, accounting for approximately 80% of aqueous egress from the normal eye. The route is from the anterior chamber angle (formed by the iris and cornea), through the trabecular meshwork, into Schlemm's canal. The latter is a 360° channel just peripheral to meshwork. It is connected to intrascleral outlet channels that take the aqueous through the sclera to reunite with the blood stream in the episcleral veins.

The uveoscleral route is less clear with regard to anatomy and physiologic significance, but probably accounts for 10-20% of aqueous outflow in the normal human eye. As with the canalicular route, the uveoscleral pathway begins in the anterior chamber angle. The aqueous is absorbed by portions of the peripheral iris, the ciliary body and probably the trabecular meshwork, from whence it passes posteriorly through the longitudinal muscle of the ciliary body to the suprachoroidal space (between the choroids and sclera). Aqueous in the suprachoroidal space may pass as far posteriorly as the optic nerve and leave the eye through a variety of emissaria around nerves and vessels in the sclera.

Filtration surgery was introduced in the first decade of the twentieth century. The basic principle is the creation of a fistula through trabecular meshwork, Schlemm's canal and sclera. Aqueous flows through the fistula to create a pool beneath the elevated conjunction (called a bleb), through which it filters to wash away in the tear film. The basic operation, in a variety of modified forms, has now been the preferred glaucoma procedure for nearly 100 years, despite serious limitations.

Limitations of filtering surgery include failure due to fibrotic closure of the fistula. Of even greater concern are the complications associated with excessive outflow, which include an intraocular pressure that is too low (hypotony) and a conjunctival filtering bleb that becomes too thin, with leakage and the risk of infection (endophthalmitis).

Drainage implant surgery was developed primarily to overcome the problem of fistula closure, since a conduit passes from the anterior chamber angle, through the fistula, to a plate beneath the conjuctiva. However, these operations are also complicated by early hypotony and late failure due to obstruction of the conduit or excessive fibrosis over the plate. There is a need, therefore, for a device and method of using same that reliably channels aqueous into pathways without creating hypotony or a filtering bleb.

Although the uveoscleral pathway may only account for 10-20% of aqueous outflow in the normal state, there is evidence that it can be enhanced to accommodate a significantly greater percentage of outflow. For example, topical prostaglandins, which work nearly exclusively by increasing uveoscleral outflow, can lower the intraocular pressure by 30-50% in some patients. Even more compelling are the results of early surgical attempts to enhance uveoscleral outflow.

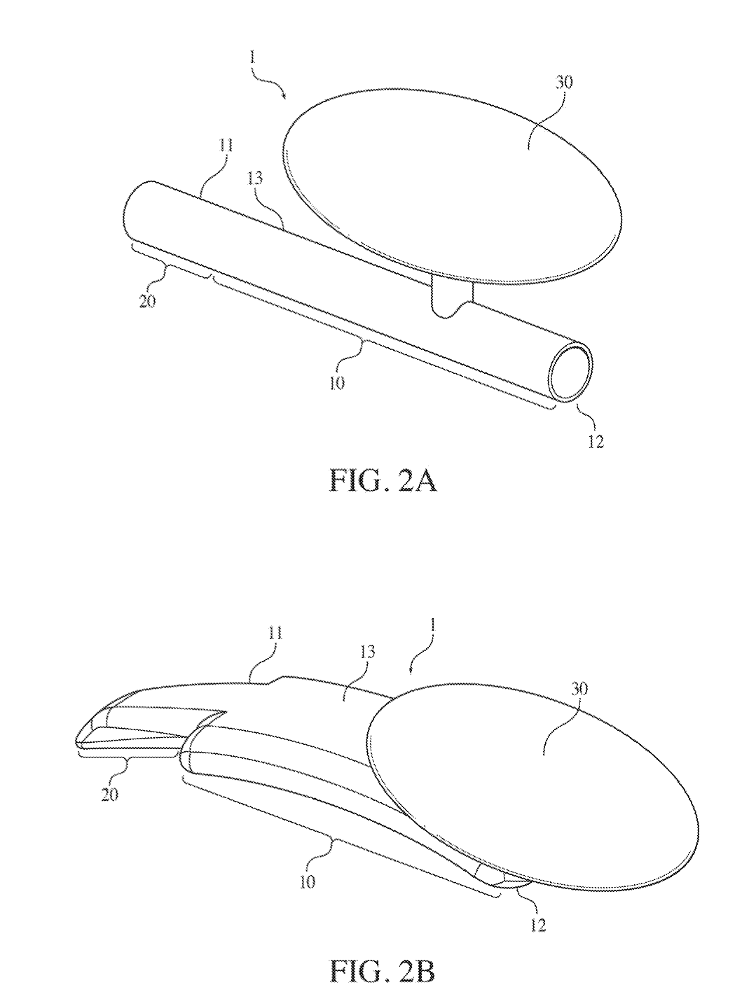

In the first decade of the twentieth century, paralleling the introduction of filtering surgery, an operation was devised to enhance uveoscleral outflow, called cyclodialysis. Referring to FIGS. 2A and 2B, the basic principle is separation of the ciliary body from the scleral spur, which provides a direct route for aqueous flow from the anterior chamber angle to the suprachoroidal space. Unlike filtering surgery, however, cyclodialysis enjoyed only limited acceptance in the twentieth century. Although it was commonly used during the first half of the century, serious limitations led to its virtual abandonment by mid-century. The limitations were two-fold. The so-called cyclodialysis cleft often worked too well with significant hypotony, and in many patients, the cleft would close suddenly, with a profound rise in the intraocular pressure.

A variety of efforts have been made to prevent closure of the cleft by wedging flaps of ocular tissue or plastic devices into the space. To date, none of these techniques have proved successful.

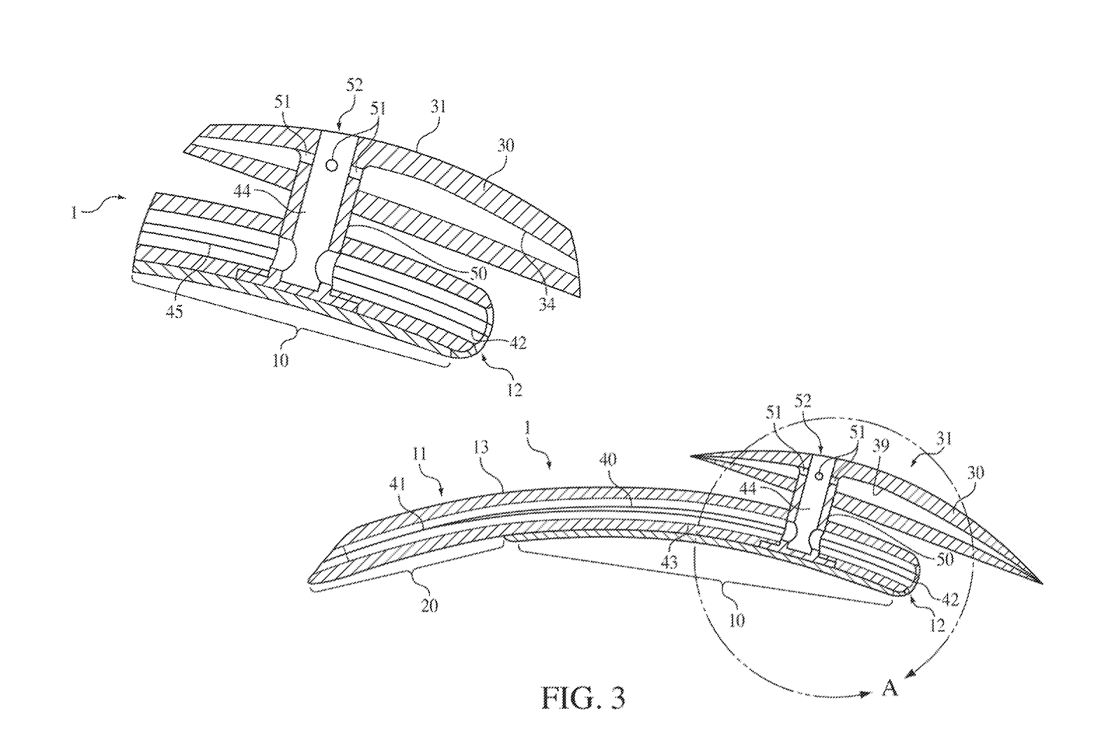

Embodiments of the invention generally relate to eye implants, more particularly, to an ophthalmic shunt and method of using an ophthalmic shunt to enhance uveoscleral drainage in the eye thereby lowering eye pressure and relieving the symptoms of various eye diseases such as, for example, glaucoma. Various embodiments of the ophthalmic shunt 1 are exemplified in FIGS. 2A and 2B, and generally include an elongate body 10 having a forward end 11 and a spaced back end 12, an insertion head or an insertion head portion 20 extending from the forward end of the elongate body and a plate 30 positioned on an upper surface 13 of the elongate body which may traverse the sclera following implantation. In such embodiments as shown in FIG. 3, the shunt 1 may include a conduit 40 having a first end 41 defined on the insertion head and a second end 42 of the conduit 40 at the back end 12 of the elongate body 10 and extending continuously through the insertion head 20 and elongate body 10. The conduit 40 may, therefore, allow fluid to traverse the shunt 1 from the insertion head 20 to the back end 12 of the shunt 1, and in particular embodiments, following implantation, the conduit 40 may carry aqueous humor from the anterior chamber of the eye to the suprachoroidal space where it may be absorbed by surrounding tissue (see FIG. 1).

In certain embodiments also illustrated in FIG. 2, the conduit 40 may be branched with first end 41 defined on the insertion head 20 and a first branch 43 extending from the forward end to the back end 12 of the elongate body 10 and providing a channel for fluid flow from the insertion head 20 and forward end 11 of the elongate body to the back end 12 of the elongate body 10, as described above. In addition to the first branch 43 in such embodiments, a second branch 44 may be provided which extends dorsally through the elongate body 10 to an upper surface 13 of the elongate body and provides a channel for flow of fluid from the insertion head and forward end of the elongate body to an upper surface of the elongate body as indicated by the arrow 45 in FIG. 3. In some embodiments, the second branch 44 may be positioned to correspond with the joint between the elongate body 10 and the plate 30, and a connector 50 may be positioned to extend the second branch 44 beyond the outer surface of the elongate body 10 such that second branch 44 of the conduit may encompass a lumen of the connector 50, thereby providing a channel for fluid flow from the insertion head 20 to the plate 30 which, following implantation, may be positioned in the anterior chamber of the eye. In some embodiments, one or more first openings 51 of the second branch 44 of the conduit 40 may be positioned on the connector 50 below the plate 51. In other embodiments, one or more second openings 52 of the conduit 40 may be positioned on an upper surface 31 of the plate 30, and in certain embodiments, one or more first openings 51 positioned on the connector 50 below the plate 30 and one or more second openings 52 positioned on an upper surface 31 of the plate 30 may be provided. Therefore, aqueous humor from the anterior chamber may be carried through the second branch 50 through the elongate body 10 to the plate 30 which when implanted may be positioned to allow the fluid to traverse the sclera and be released into the subconjunctival space. The shunt 1 of various embodiments may, therefore, provide for delivery of fluid from the anterior chamber of the eye through insertion head 20 to the back end 12 of the elongate body 10 and into the suprachoroidal space through the first branch 43 and from the anterior chamber of the eye to the subconjunctival space through the second branch 44.

The plate 30 may have any configuration or shape. For example, the plates of various embodiments may have a shape including but not limited to polygonal, rounded polygonal, circular, oval, elliptical or combinations thereof. In such embodiments, the plate 30 may be of any size. For example, in some embodiments, the diameter, width, or length of the plate may extend beyond the width of the connector by a portion of a millimeter thereby providing a flange around the upper portion of the connector, and in other embodiments, the plate may have a diameter or width/length that may extend beyond the width or length of the elongate body, as indicated in FIG. 2. In some embodiments, the plate 30 may have a symmetrical shape such as, for example, a circular, square, or diamond shape of any size. For example, in such embodiments, the plate may have a diameter or width/length of from about 0.5 mm to about 10 mm. In other embodiments, the plate may have a diameter or width/length of about 3 mm to about 9 mm, and in still other embodiments, the plate may have a diameter or width/length of about 6 mm. In other embodiments, the plate may have a diameter or width/length along a first axis that is greater than the diameter or width/length along a second axis to provide a plate having an asymmetrical shape such as, for example, an oblong, elliptical, rectangular or other asymmetrical polygonal shape.

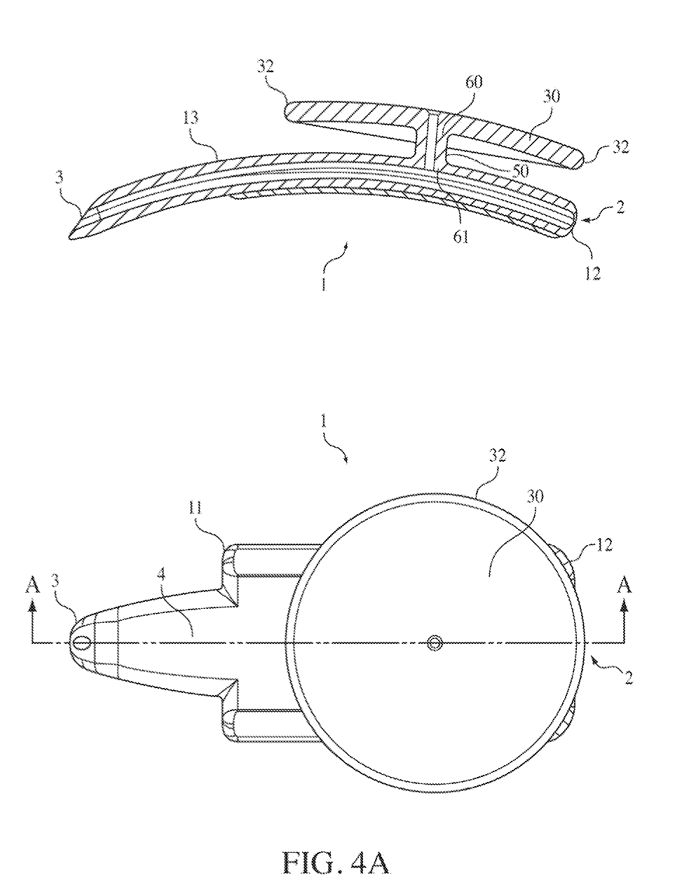

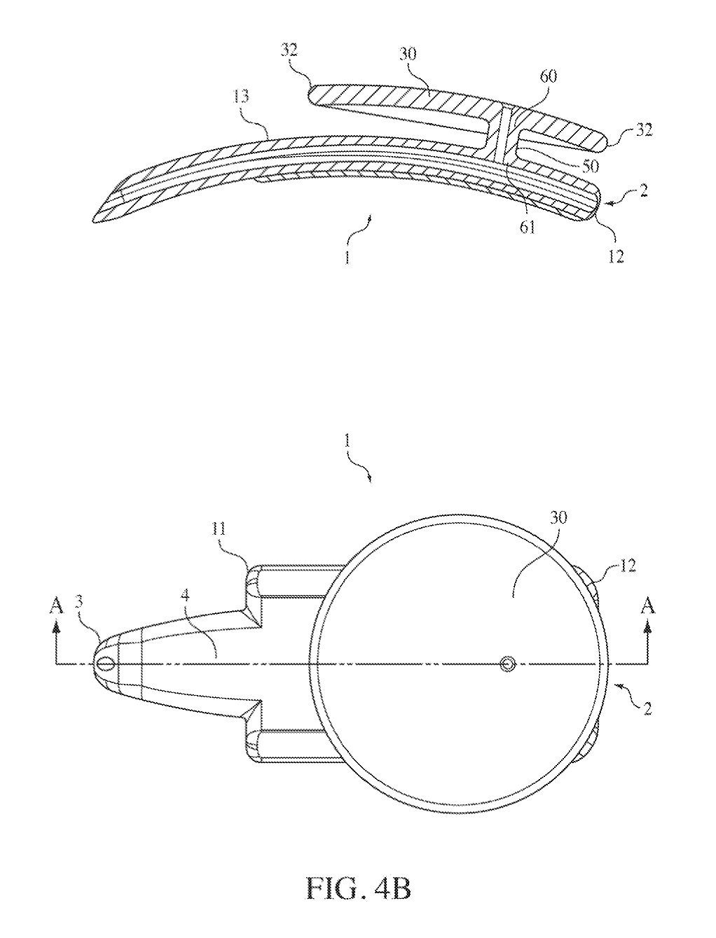

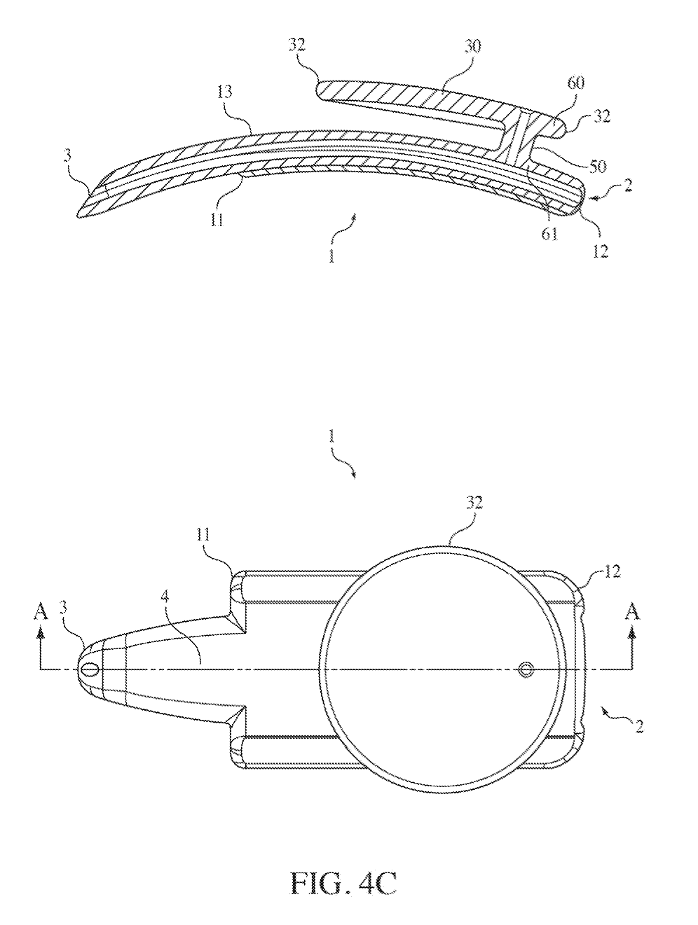

In various embodiments, the connector-plate joint 60 between the plate 30 and the connector 50 may be in any configuration. For example as illustrated in FIG. 4, in some embodiments, the connector-plate joint 60 may be at about the midpoint of the plate 30 such that the distance between the connector and the outer edge of the plate may be equal on all sides as illustrated in FIG. 4A. In the exemplary embodiment provided in FIG. 4A, a circular, 6 mm diameter plate 30 may be joined to the connector 50 at the midpoint of the plate such that the distance between the outer, circumferential edge 32 of the plate 30 and the connector is about 3 mm at every point along the circumference of the plate 30. In other embodiments as shown in FIGS. 4B and 4C, the connector-plate joint 60 may be offset from the midpoint of the plate 30. For example, in the exemplary embodiment provided in FIG. 4B, the connector-plate joint 60 between the connector 50 and a circular, 6 mm diameter plate 30 may be offset toward the anterior 2 of the device such that the connector 50 may be, for example, about 2 mm from the anterior edge of the plate 30 along the longitudinal axis 4 of the shunt 1, indicated by the dashed line in FIGS. 4A, B and C, and about 4 mm from the opposite posterior edge of the plate along the longitudinal axis 4 of the plate 30. The shortest distance between the connector 50 and the outer edge of the plate 30 in embodiments of the invention that include an offset may vary and may depend on such factors as the location of the offset and the total diameter or width/length of the plate. For example, in various embodiments, the shortest distance from the connector to the outer edge of a plate having an offset may be about 1 mm or greater or 1.5 mm or greater, and in some embodiments, from about 1 mm to about 5 mm or from about 2 mm to about 4 mm.

Embodiments of the invention are not limited by the location of the offset. For example, in some embodiments as illustrated in FIG. 4C, the connector-plate joint 60 may be offset to the anterior 3 of the device 1 such that the joint between the connector and the plate is toward the posterior of the plate 30 corresponding with the back end 12 of the elongate body 10. In other embodiments, the joint may be offset to the anterior of the plate such that the joint between the connector and the plate is toward the anterior of the plate corresponding and forward end of the elongate body. In still other embodiments, the joint may be offset laterally to either side of the longitudinal axis of the plate. Embodiments of the invention further encompass joints between the connector and the plate that are offset in two planes. For example, various embodiments, include joints between the plate and the connector that are offset along the longitudinal and lateral axis of the plate to either side of the longitudinal or lateral axis of the plate, such that, in some embodiments, the joint between the connector and the plate may be in either anterior or either posterior quadrant of the plate.

Similarly, the elongate body-connector joint 61 between the elongate body 10 and the connector 50 may be at any position on the upper surface 13 of the elongate body 10. For example, in some embodiments, the elongate body-connector joint 61 may be on the longitudinal axis 4 of the elongate body 10 at about the midpoint between the forward end 11 of the elongate body 10 and the back end 12 of the elongate body 10, as illustrated in FIG. 4A. In other embodiments, the elongate body-connector joint 61 may be offset toward the back end 12 or forward end 11 of the elongate body 10 along the longitudinal axis 4 of the elongate body 10. For example, FIGS. 4B and 4C show elongate body-connector joints 61 having varying degrees of offset toward the back end 12 of the device 1. In still other embodiments, the elongate body-connector joint may be offset laterally to either the right or left of the longitudinal axis of the elongate body, and in further embodiments, the elongate body-connector joint may be offset in two planes such that, for example, the joint may be offset along the longitudinal axis toward the forward end or back end of the elongate body and the joint may be offset laterally to one or the other side of the longitudinal axis. In such embodiments, the elongate body-connector joint may be in either anterior or either posterior quadrant of the elongate body. In particular embodiments, the connector may be offset toward the back end of the device and may be separated from the back end of the elongate body by about 0.5 mm or greater, about 1 mm or greater, about 1.5 mm or greater, or about 2 mm or greater and, in some embodiments, from about 0.5 mm to about 5 mm, or about 1 mm to about 3 mm.

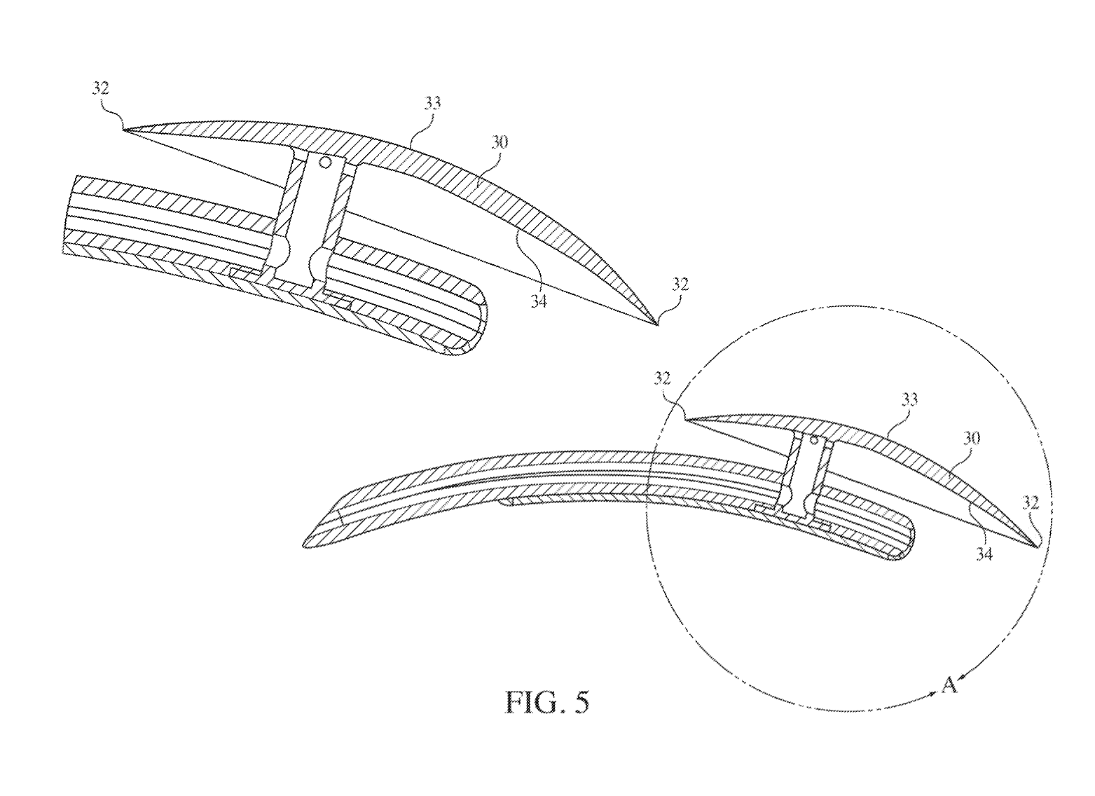

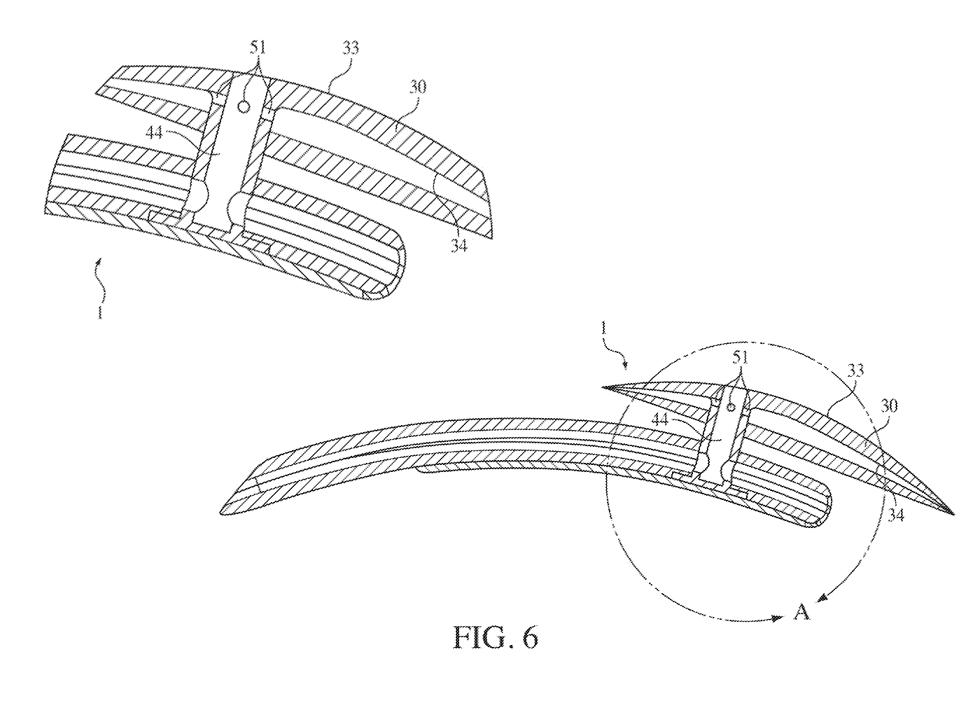

The plate of various embodiments may generally be thin, for example, in particular embodiments, the plate may have a thickness of less than about 0.1 mm. In some embodiments, the plate 30 may be substantially planar on the upper surface and/or the lower surface of the plate. For example, in some embodiments, the upper surface of the plate and the lower surface of the plate may be substantially co-planar as indicated in FIG. 4. In particular embodiments as illustrated in FIG. 5, the plate 30 may be tapered either throughout the diameter of the plate or at the circumferential edges 32 of the plate 30, and in certain embodiments, the plate may be shaped to provide, for example, an upper surface 33 that is convex. Hence, in some embodiments, the upper surface 33 may have a convex curvature and the lower surface may be substantially flat to provide a plate that has a generally fusiform shape, embodiment not illustrated. In other embodiments, both the upper surface 33 and the lower surface 34 may be curved to provide a plate 30 having a convex upper surface and a concave lower surface as illustrated in FIG. 5. In various such embodiments, the curvature of the top surface 33 may substantially match the curvature of the lower surface 34 such that the thickness of the plate is maintained or substantially maintained throughout the plate, or in some embodiments, the plate may have different curvatures or degrees of curvature on the upper 33 and lower 34 surfaces which may provide for tapered circumferential edges 32. In certain embodiments, the curvature of at least the upper surface of the plate may be substantially similar to the adjacent sclera when the shunt is implanted. Without wishing to be bound by theory, a plate having at least an upper surface that conforms to the curvature of surface of the eye or the surrounding sclera may reduce irritation to the patient receiving the implant and/or make the patient more comfortable following implantation. As illustrated in FIG. 6, in some embodiments, the curvature of the lower surface 34 may have a curvature that is steeper than the adjacent sclera. In such embodiments, a space or gap may be formed between the surface of the sclera and the lower surface 34 of the plate 30 when the shunt is implanted. Without wishing to be bound by theory, a shunt having a space or gap beneath the plate 30 may be able to accommodate liquid exiting from the second branch 44 from openings 51 located beneath the plate 30 as indicated by the horizontal arrows. The liquid, i.e., aqueous humor, that was transported from the anterior chamber through the second branch of the conduit may be retained beneath the plate at least partially filling the space or gap where it may be reabsorbed by vessels within the sclera, or when the space or gap created by the convex curvature of the plate becomes substantially or completely filled, a portion of the liquid may be forced beneath the circumferential edge and released into the subconjunctival space as pressure builds beneath the plate, as indicated by the curved arrows.

In some embodiments, the surfaces of the plate 30 and/or elongate body 10 may be substantially smooth, and in other embodiments, the lower surface 34 of the plate 30 and/or the upper surface 13 of the elongate body 10 may be textured. For example, in particular exemplary embodiments, lower surface 34 of the plate 30 and/or the upper surface 13 of the elongate body 10 may include, ridges, corrugations, bumps, fingers, concentric circles or portions of concentric circles and combinations thereof. Without wishing to be bound by theory, providing textured surfaces on the lower surface of the plate and/or the upper surface of the elongate body may increase the surface area of the lower surface of the plate and/or the upper surface of the elongate body. Such textured surfaces may stabilize the position of the shunt by allowing the plate and/or elongate body to better adhere to the sclera by providing additional surface area. Additionally, providing textured surfaces on the lower surface of the plate and/or the upper surface of the elongate body may provide channels for fluid flow or create additional space between the sclera and lower surface of the plate to facilitate egress of fluid from beneath the plate.

The plate 30 may be prepared from any material known and useful in the medical device arts. For example, in some embodiments, the plate may be prepared from a flexible material, or a flexible biocompatible polymer such as, for example, silicone, polyamide, polyethylene teraphthalate, polytetrafluoroethlyene, poly(tetramethylene succinaze) (PTMS), poly(methylmethacrylaze) (PMMA), and co-polymers thereof, and in particular embodiments, the plate may be composed of silicone. In other embodiments, the plate may be prepared from a semi-rigid or rigid material. However, without wishing to be bound by theory, it may be beneficial to prepare the plate from a flexible material to provide access to incision following implantation. For example, in some embodiments, after the shunt has been implanted, the plate 30 or a portion of the plate composed of a flexible material may be lifted or otherwise manipulated to expose the underlying incision such that it may be observed, and in particular embodiments, the exposed incision may be sutured while the plate or a portion thereof has been lifted. In other embodiments, a more rigid plate may be equipped with a hinge portion which may be positioned to allow the plate to be lifted along the hinged portion to allow the underlying incision to be exposed. The hinge portion may be prepared by any means, for example, the hinge may be a thinner region of a semi-rigid material that is more flexible than surrounding material or a second flexible material that is incorporated into a rigid or semi-rigid plate.

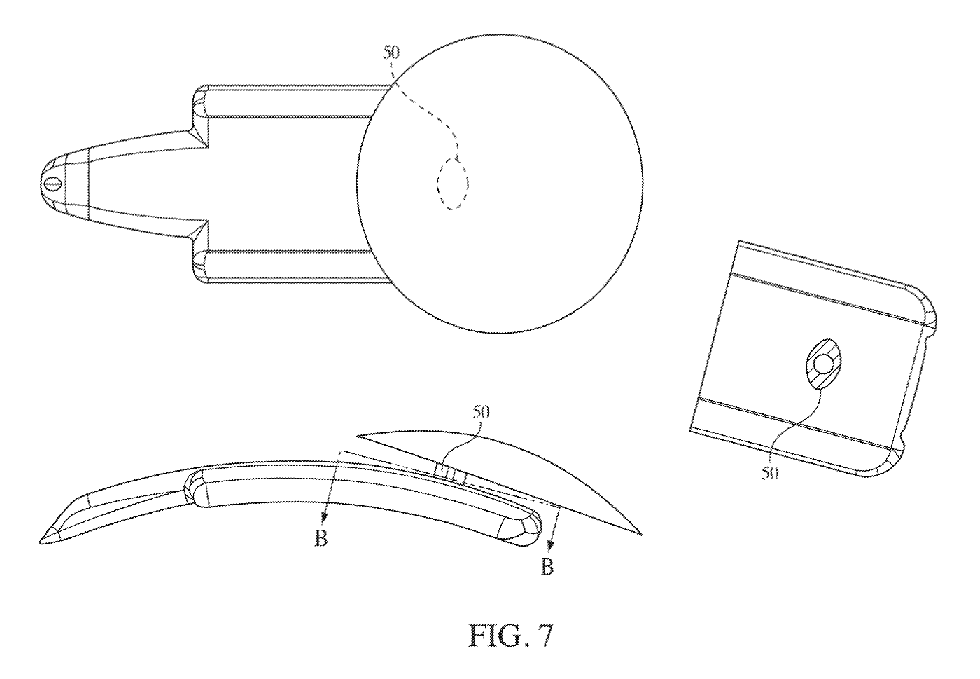

In various embodiments, the connector 50 may be a simple tube located on the upper surface 13 of the elongate body 10 which connects the plate 30 to the upper surface 13 of the elongate body 10, and in some embodiments, the connector 50 may be positioned to extend a second branch 44 of the branched conduit 40 such that the second branch 44 becomes the lumen of the connector 50. Generally, the connector may be thin, and in some embodiments, the connector may be shaped. For example, as illustrated in FIG. 7 in particular embodiments, the connector 50 may have an vesica piscis or “eye” shape. In other embodiments, the connector 50 may have a semicircular shape or an angular shape such as, for example, a square, rectangle, or triangle shape. Without wishing to be bound by theory, the shape of the connector may aid in stabilizing the shunt within the incision by, for example, limiting movement of the shunt after the incision has been sutured. The diameter of the connector 50 may similarly vary among embodiments and may be a function of the shape of the connector. For example, in some embodiments, the connector 50 may have a diameter of less than about 1 mm and, in other embodiments, less than about 0.5 mm or less than about 0.25 mm or less than about 0.1 mm. In still other embodiments, the connector 50 may have a diameter of from about 1 mm to about 0.05 mm or from about 0.75 mm to about 0.1 mm.

In various embodiments, the connector 50 may be of sufficient length to traverse the sclera of the patient when the shunt 1 is implanted, and in certain embodiments, the connector 50 may include additional length that allows the connector 50 to protrude beyond the outer surface of the sclera by, for example, about 0.01 mm to about 0.1 mm, which may provide a space beneath the plate 30 for egress of fluid. Thus, connectors of various lengths are envisioned. For example, in some embodiments, the height of the connector 50 may be from about 0.2 mm to about 1.0 mm from upper surface of the elongate body 10 to the lower surface 34 of the plate 30, and in other embodiments, the height of the connector 50 may be from about 0.5 mm to about 0.8 mm from upper surface 13 of the elongate body 10 to the lower surface 34 of the plate 30. In particular embodiments, the height of the connector 50 may be at least about 0.6 mm from upper surface 13 of the elongate body 10 to the lower surface 34 of the plate 30.

In various embodiments, the connector 50 and second branch 44 of the conduit 40 may positioned such that it is generally perpendicular to the first branch 43 of the conduit 40 and may branch from the conduit 40 within the elongate body 10 at about a right (90° angle. In other embodiments, the connector 50 and second branch 44 of the conduit 40 may extend from the conduit 40 at an angle that is greater than about 90° when measured from the portion of the conduit anterior to the connector, such as for example, from greater than 90° to about 135°.

The connector 50 may be made of any material known and useful in the medical device art. For example, in some embodiments, the connector 50 may be prepared from the same material as the elongate body 10, and in other embodiments, the connector 50 may be prepared from the same material as the plate 30. In still other embodiments, a portion of the connector 50 may be prepared from the same material as the elongate body 10 and another portion of the connector 50 may be prepared from the same material as the plate 30, and in further embodiments, at least a portion of the connector 50 may be prepared from a mixture of the material of the elongate body 10 and the plate 30. In yet other embodiments, the connector 50 may be prepared from a different material than either the elongate body 10 or the plate 30.

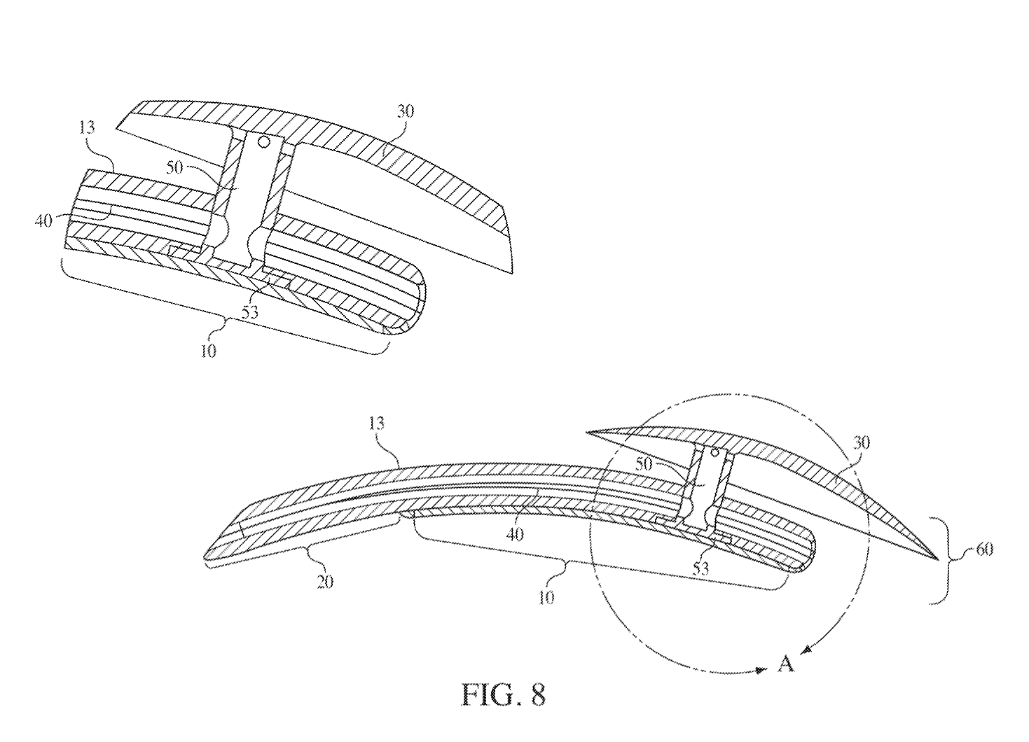

The means by which the connector 50 is coupled to the elongate body 10 and the plate 30 may similarly vary. For example, in some embodiments, the connector 50 may be molded at the same time as the elongate body 10 or the plate 30, or the connector 50 may be manufactured separately and held in place by, for example, an adhesive or a snap. For example, in some embodiments such as the exemplary embodiment provided in FIG. 8, the connector 50 may be prepared from the some material and molded at the same time as the plate 30 to create a connector-plate assembly 60. The connector 50 may further include a base portion or flange 53 which extends beyond the circumference of the connector and provides a means for attaching the connector 50 and plate 30 to the elongate body 10. In addition, the base portion or flange 53 may provide a means for limiting vertical movement of the connector 50 and plate 30 when these components are prepared from a different material than the elongate body 10 allowing the connector-plate assembly 60 to remain coupled to the elongate body 10 even when acted on by a force that would otherwise tend to separate the connector-plate assembly 60 from the elongate body 10. In further embodiments, a connector-plate assembly 60 may include more than one flange portion. For example, in some embodiments, the connector-plate assembly 60 may include a base flange 53 as depicted in FIG. 8 and an intermediate flange portion (not depicted) that contacts a portion of the elongate body 10 above the conduit 40 and/or the upper surface 13 of the elongate body 10.

Embodiments of the invention are not limited by the means by which such shunts 1 are manufactured. Therefore, any method of manufacture may be used to prepare such devices. However, in one exemplary embodiment, the shunt 1 of the invention may be prepared by molding a connector-plate assembly 60 having one or more flange portions from a first material such as, for example, silicone or any of the materials described above. The connector-plate assembly 60 may then be placed within a second mold and the elongate body 10 and, in certain embodiments, the elongate body 10 and the insertion head 20 may be molded around the connector-plate assembly 60. In such embodiments, the connector-plate assembly 60 may become an integral part of the device thereby reducing the likelihood of the connector-plate assembly 60 become dissociated from the elongate body 10, even under extreme circumstances.

In some embodiments, the connector 50 and plate 30 may merely provide a means for maintaining the position of the shunt 1 following implantation by physically attaching the shunt 1 to the sclera by placing the connector 50 in the incision and suturing around the connector 50. However, in certain embodiments, the plate 30 may further provide a secondary means for fluid flow out of the anterior chamber of the eye through the shunt and may allow extraneous aqueous humor to flow from the anterior chamber to both the suprachoroidal space through the second end 42 of the conduit and/or the subconjunctival space through one or more apertures or openings 51, 52 in the connector 50 and/or plate 30 as illustrated in FIG. 3. For example, in some embodiments, one or more apertures or openings 51 that are perpendicular to the lumen may be provided in the connector below the lower surface 34 of the plate 30 and positioned to allow fluid to flow out from the lumen of the connector 50 into a space or gap between the outer surface of the sclera and the lower surface 34 of the plate 30. The one or more apertures or openings 51 may be configured in any way. For example, in some embodiments, one opening may be provided in the connector 50 just below the lower surface 34 of the plate 30, in other embodiments, two openings may be provided on either side of the connector 50, and in still other embodiments, three, four, five six, or more spaced openings 51 may be provided around the circumference of the connector 50. In further embodiments, clusters of two or more spaced openings 51 may be provided on one or more sides of the connector 50. In other embodiments, one or more openings 52 may be provided on the upper surface 31 of the plate 30 such that fluid may flow through the connector 50 and exit the shunt 1 through the one or more openings 52 in the upper surface 31 of the plate 30 thereby allowing fluid to enter the subconjunctival space. In still other embodiments, a plurality of openings 51, 52 may be provided both on connector 50 and on the upper surface of the plate 30 to provide two means for the outlet of fluid from the second branch 44 of the conduit 40.

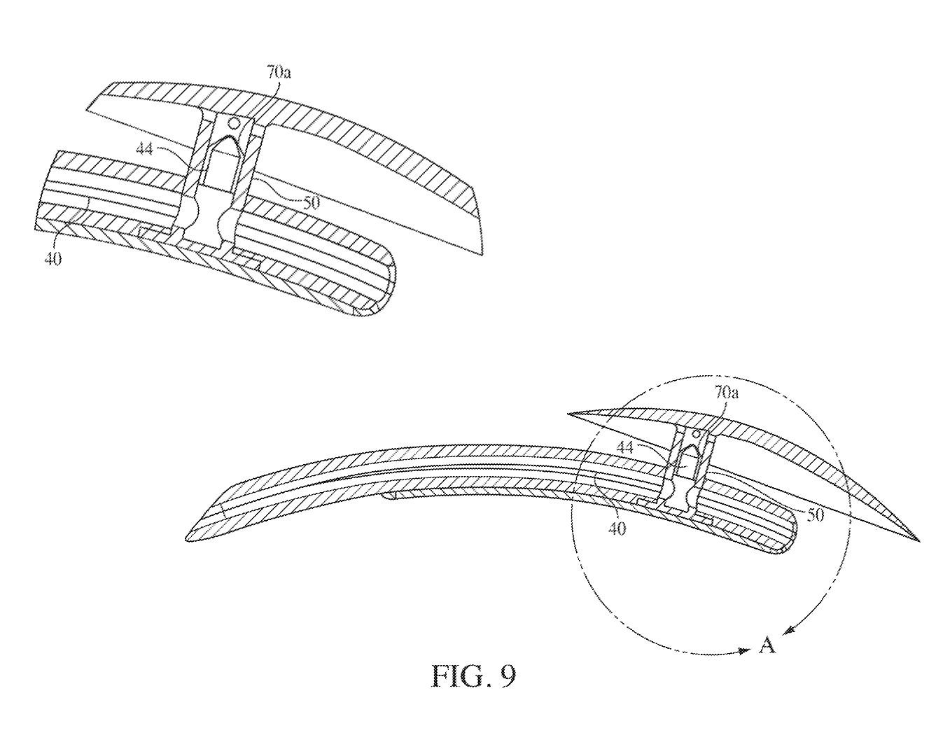

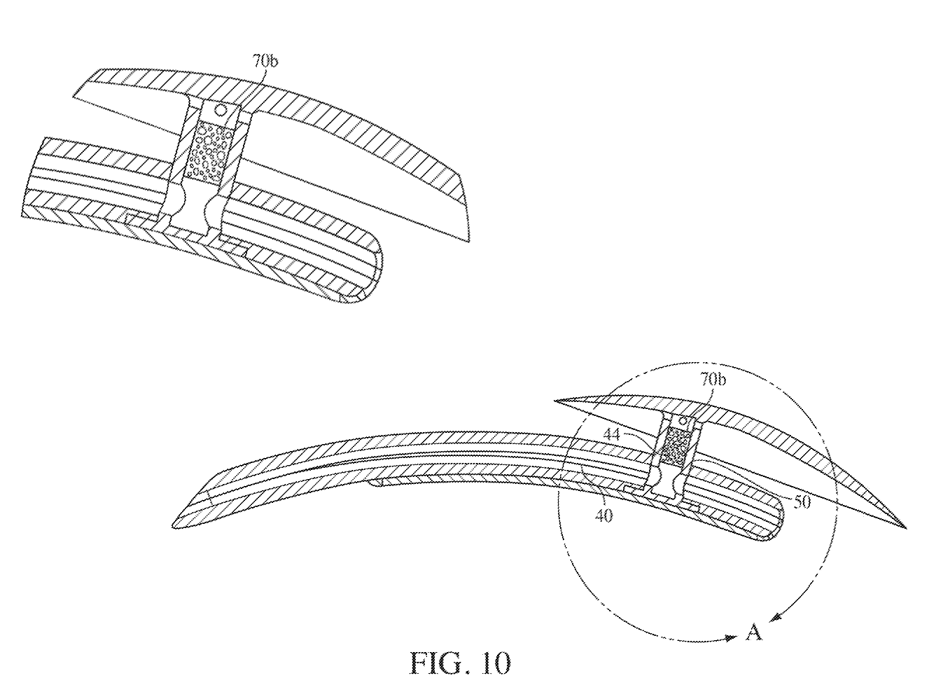

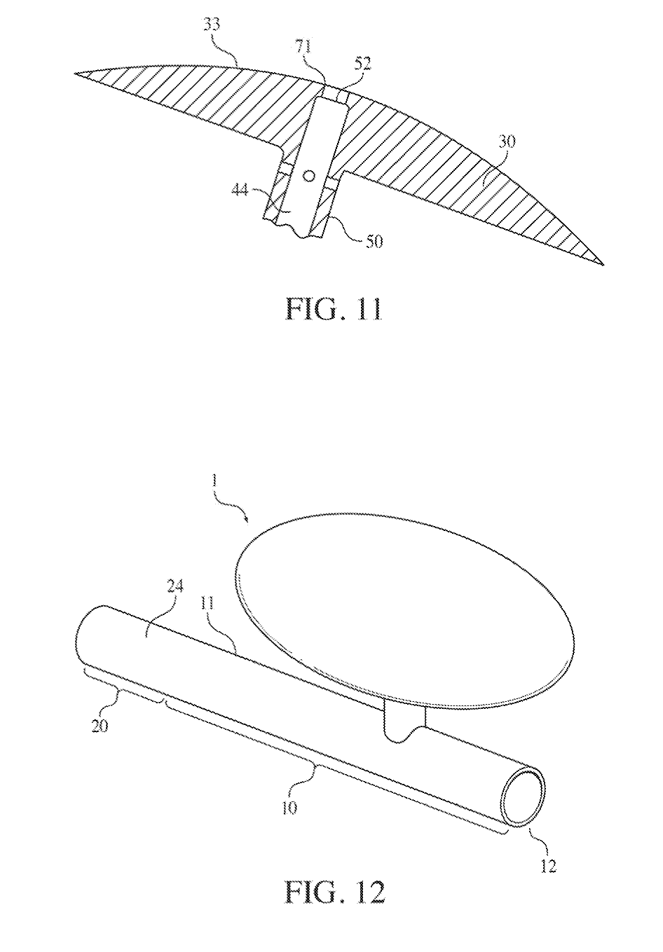

With reference to FIGS. 9 and 10, in various embodiments, the plate 30 and/or conduit 50 may be further equipped with one or more flow regulators 70 which may be positioned to provide a means to control the outlet of fluid through the second branch 44 of the conduit 40. Numerous flow regulators 70 are known in the art, and any means for regulating the flow of fluid through the connector 50 may be used including, but not limited to, various types of valves 70a, porous materials 70b, membranes, flaps, and the like and combinations thereof, and flow regulators 70 may be positioned either within the connector 50, within one or more of the apertures or openings on either the connector 51 or on an outer surface of the plate 52, or combinations thereof. In some embodiments, the flow regulator 70 may be removable. For example, in particular embodiments, a membrane that can be at least partially removed by, for instance, puncturing the membrane to increase flow through the one or more apertures or openings 51, 52 may be used as a flow regulator. One exemplary embodiment is provided in FIG. 11 which shows a membrane 71 that encompasses an opening 52 on an outer surface 33 of the plate 30. In such embodiments, the membrane 71 may be punctured to allow or increase flow of fluid through the opening 51 on the outer surface 33 of the plate 30. Puncturing such a membrane may be accomplished by any means. For example, in some embodiments the membrane may be punctured mechanically using, for example a surgical tool, and in other embodiments, the membrane may be punctured using a laser. In still other embodiments, a laser or other means may be used to partially remove or increase the pore size of a porous material positioned either on an outer surface of the plate 30 or within the connector 50, and in still other embodiments, a valve or flap 70a of FIG. 9 may be opened to increase the flow of material through the second branch 44 of the conduit 40. In yet other embodiments, the flow regulator 70 and 71 in FIG. 9-11 may be made of a biodegradable material, and flow through the second branch 44 of the conduit 40 may increase as the membrane or porous material degrades following implantation. In still other embodiments, the flow regulator may be a suture encircling the connector 50 which may cause the connector 50 to be constricted when the suture is tightened around the connector 50 thereby reducing fluid flow through the connector 50. Such a suture may be applied before or during the implantation process and may be loosened or tightened at any time during implantation or treatment to allow flow through the connector 50 to be increased or decreased, and in some embodiments, the suture may be removed to allow free flow of liquid through the connector 50.

As discussed more fully below, providing a means for adjusting or controlling flow through the second branch 44 of the conduit 40 may allow improved control over fluid pressure in the anterior chamber and/or provide a mechanism to handle an overflow of fluid, or the second branch 44 may be opened or partially opened as fluid flow through the first branch 43 of the conduit 40 is reduced by, for example, mechanical breakdown of the shunt 1 or blockage of the first branch 43 of the conduit 40.

The elongate body of various embodiments may be configured in any way. For example, in some embodiments as shown in FIG. 12, the elongate body 10 may be a tube or a flattened tube having an insertion head 20 extending from or on the forward end 11 of the elongate body and one or more openings at the back end 12 of the elongate body. In some such embodiments, the transition between the insertion head 20 may represent an extension of the elongate body 10 that is meant to be inserted into the anterior chamber of the eye, but there may be little to distinguish these elements. Thus, the junction of the insertion head 20 and the forward end 11 of the elongate body 10 may be smooth, such that the transition from one element to the other is substantially seamless. In other embodiments, there may be an obvious transition between the insertion head and the elongate body. For example, in some embodiments, a groove, channel, or furrow 24 may extend around the circumference of the insertion head 20 at the transition between the insertion head 20 and the elongate body 10 which may provide a means for sealing the shunt by allowing stretched tissue at the incision site to relax into the groove and securing the shunt 1 by acting to limit movement once implanted. In other embodiments, there may be a ridge of material extending along the circumference of the insertion head at the transition between the insertion head and the elongate body. In some embodiments flattened tube, the elongate body 10 and insertion head 20 of a tube or flattened tube elongate body 10 may have a combined length of from about 5 mm to about 15 mm. In other embodiments, such tubular elongate bodies 10 and insertion heads 20 may have a diameter of from about 200 μm to about 400 μm. In particular embodiments, tubular elongate bodies may be prepared from a pliable or semi-rigid material such that an otherwise straight elongate body may conform to the shape of the eye following implantation.

With reference to FIG. 13, in particular embodiments, the elongate body 10 may include an insertion head 20 extending generally longitudinally from the forward end 11 of the elongate body 10 which is adapted for insertion into the anterior chamber of the eye. In some embodiments, the insertion head 20 may include a shearing edge 21 constructed and arranged for cutting eye tissue engaged thereby. Embodiments of the invention are not limited by the configuration of the shearing edge of insertion head. For example, in some embodiments, the shearing edge may have a rounded or arc shape, and in other embodiments, the shearing edge may have a chisel shape, scalpel shape, and the like.

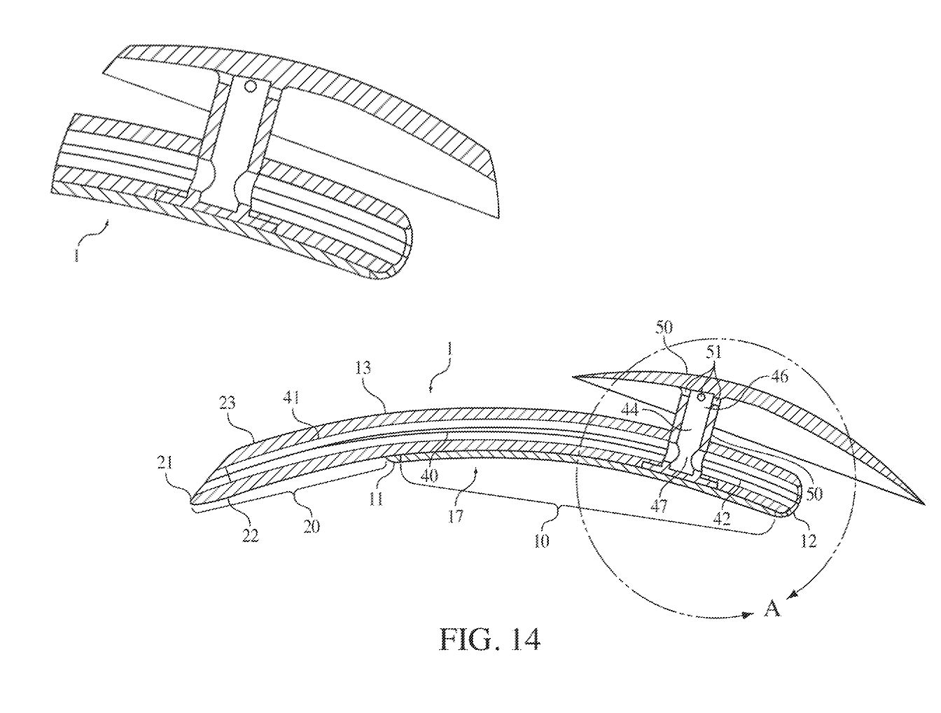

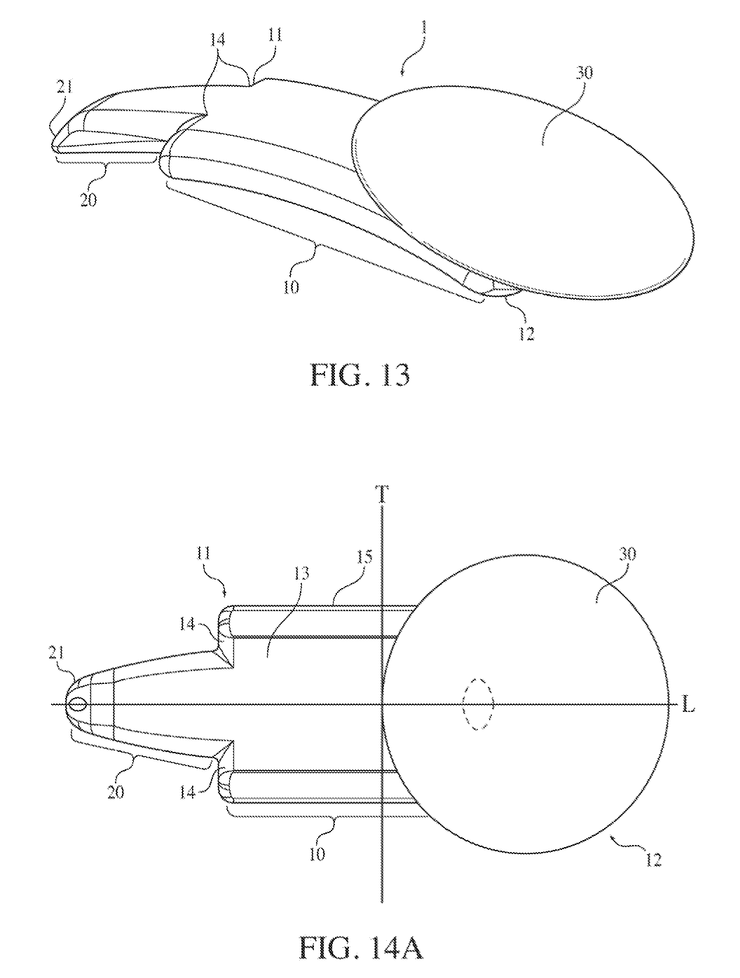

In certain embodiments, a shoulder 14 may be formed at the junction of the forward end 11 of the elongate body 10 and the insertion head 20. In such embodiments, the elongate body 10 may be configured as described in U.S. Pat. No. 7,041,077 entitled “Uveoscleral Drainage Device” and filed Jul. 21, 2003, U.S. application Ser. No. 11/347,398 entitled “Uveoscleral Drainage Device” and filed Mar. 13, 2006 or U.S. application Ser. No. 12/135,848 entitled “Uveoscleral Drainage Device” and filed Jun. 9, 2008, each of which are hereby incorporated by reference in their entireties. In particular, referring to FIG. 14, the conduit 40 which may extend from a forward end 11 of the elongate body 10 to a spaced back end 12 of the elongate body 10 along a longitudinal axis (L). The elongate body 10 may further include a first elongate edge 15 and a spaced second elongate edge 16 that extend respectively from the forward end 11 to the back end 12 of the elongate body 10 on either side and generally parallel to the conduit 40 and provide lateral boundaries of the elongate body 10. The insertion head 20 may extend longitudinally from the forward end 11 of the elongate body 10 and a shoulder surface 14 may be formed at the junction between the elongate body 10 and the insertion head 20. In such embodiments, the surface of the shoulder 14 may extend laterally at a substantially right (90° angle and to either side of the longitudinal axis (L) of the elongate body 10. Thus, the shoulder 14 may provide a surface that is perpendicular to the insertion head 20 and may provide a means for limiting movement of the elongate body 10 through an incision made by the shearing edge 21 of the insertion head 20.

In some embodiments, the base portion 22 of the insertion head 20 may extend in a substantially co-planar manner to a lower surface 17 of the elongate body 10. Alternatively, the insertion head 20 may extend from a portion of the forward end 11 of the elongate body 10 such embodiments that insertion head 20 is substantially the same thickness as the shoulder surface from the upper portion 23 to the base portion 22 of the insertion head 10. The shoulder surface 14 may, therefore, extend about the periphery of the insertion head 20. In certain embodiments, the thickness of the insertion head 20 may increase from the forward most shearing edge 21 of the insertion head 20 to the junction with the shoulder surface 14. For example, in some embodiments, the thickness of at least a portion of the insertion head 20 at the junction with the shoulder 14 may be substantially equal to the thickness of the elongate body 20.

In some embodiments, the junction of the insertion head 20 against the forward end 11 of the elongate body 10 may define a shoulder surface 14, and in particular embodiments, the insertion head 10 may be tapered such that the width of the insertion head 10 decreases at the junction of the elongate body 10 and the shoulder surface 14 to the forward most portion of the shearing edge 21. The width of the insertion head 10 at the shoulder 14 may vary among embodiments. For example, in some embodiments, the width of the insertion head 10 at the shoulder may be at least 50% of the width of the shoulder 14 as illustrated in FIGS. 14A and 14B. In other embodiments, the width of the shoulder may be about 75% or about 80% to about 50% of the width of the shoulder, and in still other embodiments, the width of the insertion head may be about 10% or about 25% to about 50% of the width of the shoulder. In some such embodiments, the insertion head 10 may be center along the forward edge 11 of the elongate body 10 and thus, may be centered along the longitudinal axis (L) of the elongate body 10 as illustrated in FIGS. 14A and 14B. In other such embodiments, the insertion head may be off set to one side or the other, i.e., either side of the longitudinal axis (L) of the forward end of the elongate body. Similarly, the width of the forward most portion, shearing edge 21 of the insertion head 20 may vary among embodiments and may be, for example, a sharp point, a straight edge or combination of two or more straight edges combined at one or more angles, a chiseled edge, a curved edge or combination thereof, and in such embodiments, the width of the leading edge may be, for example, from about 50% of the width of the shoulder to about 25%, 10%, 5% or less of the width of the shoulder.

Without wishing to be bound by theory, the taper of the insertion head 20 may allow the insertion head 20 to seal the incision made by the shearing edge 21 between the anterior chamber and the suprachoroidal space. In other embodiments, the insertion head 20 can have a shape that acts to dilate tissue as it is inserted into position. This may cause the tissue to stretch around the exterior surface of the insertion head 20 such that the incision may be self-sealing against the insertion head 20, and in certain embodiments, a portion of the insertion head 20, spaced from the shearing edge 21, may define a circumferentially extending groove or waist that is configured such that the stretched tissue can relax fractionally to both seal and fixate the shunt 1 relative to the incision. In such embodiments, the groove may be at any position on the insertion head 20, and in some embodiments, the groove may correspond with the junction between the shoulder surface 14 and the insertion head 20. Additionally, in some embodiments, the shoulder surface 14 of the elongate body 10 may be adapted to engage tissue portions separating the anterior chamber and the suprachoroidal space such that when the tissue portions are so engaged, the shoulder surface 14 may act to further seal the incision made by the shearing edge 21 of the elongate body 20. The shoulder surface 14 may also aid in limiting anterior movement or displacement of the shunt 1 after implantation, which may help prevent the forward end 11 of the elongate body 10 and/or the shoulder surfaces 14 from penetrating into and entering the anterior chamber of the eye.

The elongate body 10 of various embodiments may generally be thin to provide a less irritating fit within the eye. For example, in some embodiments, the elongate body may be up to about 1.5 mm thick, and in other embodiments, the elongate body may have a thickness of from about 0.25 mm to about 1.0 mm. The elongate body of various embodiments may have a length from the forward end 11 to the back end 12 sufficient to extend from proximate the interior surface of the anterior chamber to the suprachoroidal space of the eye, and the length of the elongate body 10 may vary based on the age and/or size of the individual into whom the device is to be implanted. Various embodiments of the invention encompass elongate bodies 10 having any numerous lengths and thicknesses which may be necessary for proper implantation into any individual. For example, in some embodiments, the elongate body 10 may have a length of from about 5 mm to about 10 mm, and in other embodiments, the elongate body 10 may have a length of from 6 mm to 8 mm. In still other embodiments, the length of the elongate body 10 may be 5 mm, 6 mm, 7 mm, 8 mm, 9 mm or 10 mm.

The shape of the elongate body 10 along the longitudinal axis (L) may be adapted to extend along a portion of the curvature of the sclera of the eye. Thus, in various embodiments, the elongate body 10 may have a substantially planar shape or an arcuate shape along at least a portion of its length, and in some embodiments, one or more portion of the elongate body 10 may be substantially planar and one or more other portions of the elongate body 10 may have an arcuate shape. In such embodiments, the arcuate portion of the elongate body 10 may have various circumferences such that the elongate body may maintain a smooth outer surface.

The elongate body 10 may also have a variety of cross-sectional shapes. For example, in some embodiments, lateral axis (T) of the elongate body 10 may have a substantially planar shape and in others, the lateral axis (T) may have an arcuate shape or a combination of one or more substantially planar portions and one or more substantially arcuate portions. In particular embodiments, the elongate body 10 may have a substantially fusiform cross-sectional shape such that the elongate body is tapered toward the first elongate edge 15, the spaced second elongate edge 16, or both the first elongate edge 15 and the second elongate edge 16. In some embodiments, the upper surface 13 or lower surface 17 of an elongate body 10 having a substantially fusiform shape may be curved to provide a substantially convex surface about the lateral axis (T) and the opposite surface may be substantially planar about the lateral axis (T). For example, in one exemplary embodiment, the upper surface 13 of the elongate body 10 may have a substantially convex surface and the lower surface 17 may be substantially planar. In other embodiments, the upper surface 13 and the lower surface 17 of the device may be curved to produce a convex or concave surface about the lateral axis (T). In another exemplary embodiment, the upper 13 and lower 17 surfaces of the fusiform elongate body 10 may be substantially convex to create a flattened football shaped fusiform elongate body 10, and in another exemplary embodiment, the upper surface 13 of the elongate body 10 may be curved to create a convex surface and the lower surface 17 of the elongate body 10 may be curved to create a concave surface that is less steep than the upper surface 13. Without wishing to be bound by theory, an elongate body 10 having a substantially fusiform shape may aid in stabilizing the device once implanted as tissues of the eye surrounding portions of the exterior surface of the elongate body 10 are similarly curved.

In various embodiments, the back end 12 of the elongate body 10 may be continuous with the upper 13 and lower 17 surfaces and first elongate edge 15 and a spaced second elongate edge 16 and may be adapted for insertion within the suprachoroidal space of the eye. The back end 12 of the elongate body 10 may have any shape. For example, in some embodiments, the back end 12 of the elongate body 10 may include a surface that is substantially parallel to the shoulder surface 14 at the forward end 11 of the elongate body 10 and may have a thickness substantially the same as the width of the elongate body 10 between its upper 13 and lower 17 surfaces. In such embodiments, the back end 12 may be blunt, squared, squared with rounded edges, or rounded from the upper surface 13 to the lower surface 17 or from the first elongate edge 15 to the second elongate edge 16 or a combination thereof. In other embodiments, the back end 12 may be tapered or sloped to form a back end 12 which may have a chisel shape, scalpel shape, and the like. In such embodiments, the edge of the tapered or sloped back end may be sharpened, dull or rounded, and in particular embodiments, the back end 12 may be fashioned such that tissue contacted by the back end 12 of the elongate body 10 is not cut by the back end 12 of the elongate body 10 when the shunt 1 is implanted.

The conduit 40 of various embodiments, may include a first end 41 and a spaced second end 42, and in particular embodiments, the conduit 40 may include a third end 46 positioned on the connector and/or the plate of the shunt. In some embodiments, the first end 41 of the conduit 40 may be positioned at the forward end 11 of the elongate body 10, and in other embodiments, the first end 41 of the conduit 40 may be positioned on the insertion head 20 such that at least a portion of the conduit 40 is positioned on or within the insertion head 20. For example, in some embodiments, a portion of the conduit 40 may be defined on a portion of a top surface 23 of the insertion head 20, and in other embodiments, a portion of the conduit 40 may be positioned within an insertion head 20 having a tapered configuration where the thickness of the insertion head 20 is tapered from the shoulder 14 to the shearing edge 21 as illustrated in FIGS. 14 and 14A. In such embodiments, the remaining portion of the conduit 40 may be defined within the elongate body 10 and may extend from the forward end 11 to the back end 12 of the elongate body 10. In embodiments in which the first end 42 of the conduit is positioned on the insertion head 20, the first end may be defined on any part of the insertion head 20. For example, in some embodiments, the first end 11 may be defined posterior to the shearing edge 21, toward the forward end 11 of the elongate body 10. In other embodiments, the first end 11 of the elongate body 10 may be defined between the shearing edge 21 and about the midpoint of the insertion head 20, and in still other embodiments the first end 41 of the conduit 40 may be defined between about the midpoint of the insertion head 20 and the junction between the insertion head 20 and the forward end 11 of the elongate body 10. In certain embodiments, the first end 41 of the conduit 40 may be located at or near the shearing edge 21 of the insertion head 20. In additional embodiments, the first end 42 of the conduit 40 may be positioned to the right or left of a longitudinal axis (L) of the insertion head 20 such that the first end 41 of the conduit 40 may be offset from the longitudinal axis (L) of the insertion head 20.

In further embodiments, the conduit 40 and/or the first end 41 of the conduit 40 may be tapered or otherwise configured to be received by the insertion head 20, and in still further embodiments, the first end 41 of the conduit 40 may be spaced from the shearing edge 21 and spaced from the shoulder surface 14 of the body 10 such that tapering may not be necessary. For example, in one exemplary embodiment, the first end 41 of the conduit 40 may be positioned at an acute angle with respect to the top surface 23 of the insertion head 20, and in another exemplary embodiment, the conduit 40 or the first end 41 of the conduit 40 may be tapered to match the taper of the insertion head 20.

In some embodiments, the conduit 40 may be a straight channel or substantially straight channel having a consistent diameter throughout the elongate body 10. In other embodiments, the conduit 40 may be tapered such that the second end 42 of the conduit 40 has a larger diameter than the first end 41 of the conduit 40, and in particular embodiments, the second end 42 of the conduit 40, as well as at least a portion of the conduit 40 leading to the second end 42, may be flattened to create a second end 42 having an oblong or oval shape. In such embodiments, the second end 42 of the conduit 40 may terminate in a broadened outflow path in free fluid communication with the suprachoroidal space. In other such embodiments, the outflow path may by in fluid communication with a hydrogel, hydrocolloid, or other absorbent material which may be either implanted separately or housed within a portion of the back end 42 of the conduit 40. In still other embodiments, a tapered conduit 40 may be fashioned to receive an operating instrument such as, for example, forceps or an obturator, as defined below. Thus, the second end 42 of the conduit 40 may further include notches or grooves, to accommodate the operating instrument and/or prevent slippage or rotation of the shunt 1 during implantation.

As described above, in various embodiments, the third end 46 of the conduit 40 may include apertures or openings 51, 52 positioned on the connector 50 or on an upper surface 31 of the plate 30. Therefore, the third end 46 of the conduit 40 may include one or more openings 51 into the lumen of the connector 50 and/or one or more openings 52 through the upper surface 31 of the plate 30. A second branch joint 47, which may deliver fluid to the third end 46 of the conduit 40, may be positioned on any portion of the conduit 40. For example, in some embodiments, the second branch joint 47 may be positioned in the middle of the elongate body 10, and in other embodiments, the second branch joint 47 may be between the forward end 11 of the elongate body 10 and about the middle of the elongate body 10. In still other embodiments, the second branch joint 47 may be between the middle and the back end 12 of the elongate body 10, and in particular embodiments, the second branch joint 47 may be positioned in the back third of the elongate body 10 as depicted in FIGS. 14 and 14A. For example, in some embodiments, the second branch joint 47 may be about 1 to about 6 mm from the back end 12 of the elongate body 10, and in other embodiments, the second branch joint 47 may be about 2 mm to about 4 mm from the back end 12 of the elongate body 10.

In various embodiments, the second branch joint 47 may a “T” type joint such that the second branch 44 diverges from the conduit 40 at about a 90° angle as illustrated in FIGS. 14 and 14A. However, in some embodiments, the second branch 44 may diverge from the conduit 40 at an angle other than a 90° angle such as, for example, an angle between 60° and 90° or 45° and 90°. As such, in some embodiments, the second branch joint 47 and the connector 50 may be aligned with each other such that the second branch 44 is straight between the second branch joint 47 and the third end 46 of the conduit 40 and the connector 40 is at about a 90° angle to the upper surface 13 of the elongate body 10. In other embodiments, where the second branch joint 47 may be at an angle other than a 90° angle, the second branch 44 of the conduit 40 may meet the conduit 40 at an angle. In such embodiments, the connector 50 may be continuous with the second branch 44 such that the connector 50 is angled with regard to the upper surface 13 of the elongate body 10, or a second joint may be positioned at the junction between the second branch joint 47 and the connector 50 such that the connector 50 may meet the upper surface 13 of the elongate body 10 at about a 90° angle.

In some embodiments, the conduit 40 may be formed as a separate element from the elongate body 10 and/or insertion head 20, such that the conduit 40 is inserted into the elongate body 10 and/or insertion head 20 after molding, or the elongate body 10 and/or insertion head 20 may be molded around the conduit 40. In such embodiments, a longitudinally extending bore may extend through the elongate body 10 such that a proximal end of the bore may be defined in the forward end 11 of the elongate body 10 and positioned adjacent to the insertion head 20. At least a portion of the conduit 40 may be positioned within the bore of the elongate body 10 such that the second end of the tube may be positioned proximate to a distal end of the bore and back end 12 of the elongate body 10. In some embodiments, a portion of the conduit 40 may extend through the proximal end of the bore and overlay a portion of the insertion head 20 or extend through a bore through the insertion head 20 that is continuous with the bore of the elongate body 10 such that the first end 41 of the conduit 40 may be positioned on or within the insertion head 20. In certain exemplary embodiments, the first end 41 of the conduit 40 may be spaced from both the shearing edge 21 and the shoulder surface 14 of the elongate body 10, and in other exemplary embodiments, the first end 41 of the conduit 40 may be located at the shearing edge 21 of the insertion head 20. In other embodiments, the conduit 40 may be integral to the elongate body 10 and may be formed integrally when the elongate body 10 is molded to create a bore/conduit having a similar arrangement to that described above.

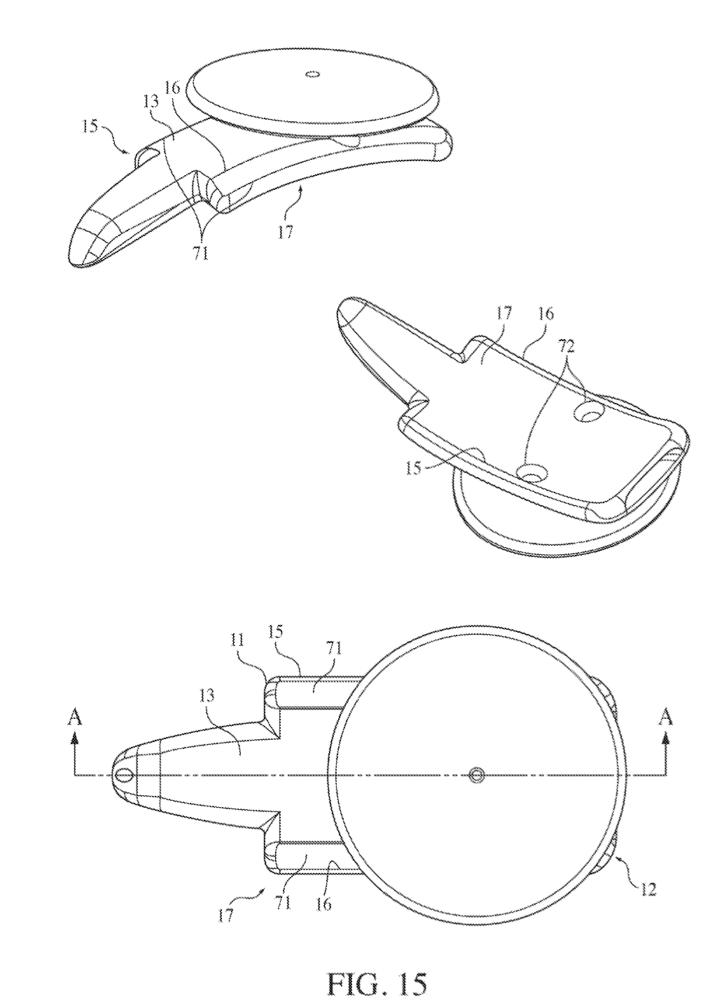

The shunt 1 of various embodiments, may further include any number of additional features that facilitate handling, implantation, stability and the like. For example, in some embodiments, as shown in FIG. 15, the shunt 1 of the invention may include one or more longitudinally extending slits 71 which may be defined on an upper surface 13, as depicted, and/or lower surface 17 of the elongate body 10. In some embodiments, the slits 71 may extend from the forward end 11 to the back end 12 of the elongate body 10, on the upper 13 and/or lower surface 17 of the elongate body 10, and in other embodiments, one or more slits 71 may extend from the forward end 11 of the elongate body 10 to the back end 12 of the elongate body 10 on one or both elongate edges 15, 16. In other exemplary embodiments, the elongate body may include one or more planar surfaces constructed and arranged for grasping by the surgical tool. Such planar surfaces may be defined on a portion of the upper surface 13 of the elongate body 10 and/or lower surfaces 17 of the elongate body 10, or such planar surfaces may be provided within the conduit 40 or on one or the other elongate edge 15, 16 of the elongate body 10. In still other embodiments, the elongate body 10 may include a combination of the slits and planar surfaces. For example, a portion of a slit in the elongate body 10 may form a planar surface. In some embodiments, a first longitudinally extending groove and second longitudinally extending groove or a planar surface may be defined on the opposite upper 13 and lower 14 surfaces of the elongate body 10 spaced respective to facilitate secure grasping of the device. Such slits and/or planar surfaces may provide a means for grasping the elongate body 10 during implantation with, for example, a surgical tool such as forceps and the like.

In other embodiments, the shunt 1 may include a wicking member or valve such as, for example, a leaflet valve, may be constructed and arranged to regulate flow of fluid from the first end 41 of the conduit 40 to the second end 42 of the conduit 40, and in certain embodiments, the wicking member or valve may be employed to control the flow of aqueous from the anterior chamber to the suprachoroidal space and/or the subconjunctival space. For example, in some embodiments, the wicking member may be positioned within at least a portion of the conduit 40, and in other embodiments, the wicking member may overlay a portion of the top surface 23 of the insertion head 20. The wicking member or valve may be positioned within the conduit 40, the first branch 43 of the conduit 40 or the second branch 44 of the conduit 40, and in some embodiments, more than one wicking member or valve may be positioned within the conduit 40, the first branch 43 of the conduit 40 or the second branch 44 of the conduit 40 to control flow through the various branches simultaneously. In one exemplary aspect, the valve may be positioned proximate the back end 12 of the elongate body 10, and therefore, proximate the second end 42 of the conduit 40. In additional embodiments, the conduit 40 itself may act to regulate the flow of fluid through the elongate body 10. For example, a hollow or empty conduit can act as a flow restrictor if properly sized. Further embodiments contemplate that proper sizing of the conduit 40 may be unnecessary as the flow may be limited by the absorptive capacity of the connective tissue surrounding the implanted device. In yet other embodiments, the shunt 1 may have a conduit 40 with an initial width that may be modified by an intervention procedure following implantation to enlarge or reduce the capacity of the conduit 40. For example, in some exemplary embodiments, a laser may be used to size the conduit after implantation.

In certain embodiments shown in FIG. 14, the shunt 1 may have one or more stitching loops, notches, bores, or suture holes 72 or the like defined in the elongate body 10 such that sutures may be passed through the loop and secured to the sclera by passing a suture through a loop, notch, bore, or suture hole and the sclera thereby securing the elongate body 10 to the sclera. For example, in some embodiments, the elongate body 10 may include a pair of spaced notches defined on either side elongate edge 15, 16 of the elongate body 10 that are constructed and arranged for facilitating suturing of the elongate body 10 to eye tissue. Such notches or bores may have a variety of shapes. For example in some embodiments, the notches or bores may be circular, semi-circular or oblong and in other embodiments the notches may have a keyhole shape. In such embodiments, the stitching loops, notches, bores, or suture holes 72 may extend between the upper 13 and lower 17 surfaces and may have at least a pair of spaced bores 72 extending through the thickness of the elongate body 10. In particular embodiments, to simplify the surgical procedure, a suture may be preloaded into a stitching loop, notch, bore, or suture hole 72 of the shunt 1 prior to implanting the device into the eye.

The stitching loops, notches, bores or suture holes 72 may be positioned at any location on the elongate body 10. However, in certain embodiments, the loops, notches, bores or suture holes 72 may be positioned a substantial distance from the back end 12 of the elongate body 10. For example, in some embodiments, the loop, notches, bores or suture holes 72 may be positioned between about 2.5 mm to about 4 mm from the back end 12 of the elongate body 10, and in other embodiments, the loop, notches, bores or suture holes 72 may be positioned at least about 2 mm from the back end 12 of the elongate body 10. In still other embodiments, the loop, notches, bores or suture holes 72 may be positioned about 3 mm from the back end 12 of the elongate body 20. Without wishing to be bound by theory, the position of the loop, notches, bores or suture holes 72 may reduce the incidence of, for example, fibrosis by removing the sutures for attaching device to the eye from sutures necessary for closing the incision. For example, in one embodiment, the device may be placed in the eye such that the incision in the eye is about 2 mm to about 2.5 mm from the back end 12 of the elongate body 10 such that the sutures associated with the loop, notches, bores or suture holes 72 are separated from the incision by about 0.5 mm to about 1.5 mm.

In further embodiments, the elongate body 10 or portions thereof and/or plate 30 or portions thereof may include an adhesive that has been applied to one or more surfaces such as, for example, the upper surface 13 elongate body and/or the lower surface 34 of the plate 30. In such embodiments, the adhesive may bond to tissue surrounding the shunt 1 thereby securing the shunt 1 in place. In some embodiments, the adhesive may be applied during the surgical procedure prior to implantation, and in other embodiments, portions of the shunt 1 may include a pre-applied adhesive that can be covered by a removable backing, which covers the adhesive during implantation and can be removed exposing the adhesive once the shunt has been implanted.

In other embodiments, the shunt 1 may include one or more barbs that allow the insertion head 20 to enter tissue by folding against the surface of the insertion head, but prevent the insertion head 20 from backing out of the tissue by extending and embedding into tissue contacted by the insertion head. Such barbs may be placed on any surface on the shunt 1 including, for example, the insertion head 20, the elongate body 10 or the lower surface 34 of the plate 30. For example, in some embodiments, a plurality of barbs may be placed over one or more surface of the elongate body 10, plate 30, or insertion head 20 such that each individual barb becomes embedded in the surrounding tissue during implantation. In other embodiments, each barb may be placed opposite a bore or cavity on an opposing structure. In such embodiments, a barb placed, for example, on an upper surface 13 of the elongate body 10 may extend through the tissue such that a tip of the barb comes to rest in a bore or cavity on a portion of the plate 30 opposing the portion of the elongate body 10 including the barb.

In further embodiments, the shunt 1 may be coated with, for example, one or more anticoagulant such as hyaluron, heparin, phosphorylcholine, butylmethacrylate and the like to encourage an aqueous boundary layer between the implant and host tissue. It is further contemplated that the absorptive capacity of the tissue surrounding the device can be influenced by the choice of biomaterials from which the shunt 1 may be made. In such embodiments, the absorptive capacity of the tissue surrounding the device may be influenced by surface area of the shunt. For example, within a fixed volume constraint, surface area may be enlarged by geometrical features or textures on the surface of the shunt such as, for example, fins, scales, fingers, corrugations, and the like.

In an alternative embodiment, the second end 42 and/or the third end 46 of the conduit 40 may include one or more flattened, flexible tubes which is configured to open when the anterior chamber pressure has risen to a level sufficient to cause the tube to open. In various embodiments, the flattened, flexible tube may be impermeable, permeable, or semi-permeable to aqueous fluid, and in some embodiments, the flattened, flexible tube may be perforated having a plurality of holes or slots into the interior lumen of the flattened, flexible tube to allow fluid to pass out of the tube. In other embodiments, the posterior portion of the tube may be split to create a plurality of capillary-like filaments or hollow tubes which may allow fluid to flow through the capillary-like filaments or hollow tubes, and in still other embodiments, the flattened, flexible tube may terminate in a plurality of filaments or wires that are configured to allow fluid to flow in spaces formed between the filaments or wires. In such embodiments, the capillary-like filaments, hollow tubes, filaments or wires may move relative to each other and against each other and may be self-cleaning in the process. In still other embodiments, when a flattened, flexible tube is constructed from a permeable or semi-permeable material, the end of the permeable or semi-permeable tube may be sealed such that fluid flow is directed through the material of the tube and not through the end of the tube. Thus, closed tubes may regulate fluid flow by selecting a permeable or semi-permeable material with an appropriate fluid flow rate through the material.

In further embodiments, the second end 42 or third end 46 of the conduit 40 may be positioned to abut or otherwise connect with a biocompatible element. For example, in some embodiments, the biocompatible element may be an absorbent, and in other embodiments, portions of the biocompatible element may be formed from impermeable, permeable, or semi-permeable material that may be shaped as a membrane, collection of fibers, or perforated sheet-like material. In still other embodiments, the biocompatible element may include shaped elements and/or geometrical features such as fins, scales, fingers, corrugations, or other textured elements that may increase the surface area of the biocompatible element to increase exposure of adjacent tissues to fluid exiting the device thereby increasing the absorptive capacity of the shunt.

In additional embodiments, the second end 42 or third end 46 of the conduit 40 may define the reservoir. For example, in some embodiments, the second end 42 of the conduit 40 may include a reservoir that is substantially or partially open to the choroid when it is operatively positioned within the eye. In such embodiments, when the ocular pressure is sufficiently elevated, the choroid may be deflected and allow fluid to pass from the reservoir and into the suprachoroidal space. In other embodiments, the reservoir may include a valve proximate the second end 42 of the conduit 40 and configured to open and allow fluid to exit the reservoir in the shunt to the suprachoroidal space when the ocular pressure is sufficiently elevated.

In still further embodiments, one or more additional drainage holes may be located on all or some of the surface of the shunt such that the one or more additional drainage holes are in fluid communication with a conduit. In such embodiments, the additional drainage holes in combination with a recessed flow path may be utilized such that opposing tissue does not occlude the flow path.

In another embodiment, the shunt may include a coiled spring that may be mounted proximate the second end 42 of the conduit 40. In this aspect, the coils of the spring may be configured to move relative to each other and against each other. The coils may be self-cleaning in the process. The coils allow the passage of fluid between them and out of the second end of the conduit.

In various embodiments, the elongate body 10 may be substantially rigid or may be substantially resilient or semi-rigid or flexible and may be made from any biological inert or biocompatible materials such as, for example, metals, ceramics, or polymeric materials. For example, in some embodiments, a biocompatible material may be a biocompatible metal, such as, gold, platinum, nickel, molybdenum, titanium, and various biocompatible metal alloys and the like. In other embodiments, the biocompatible material may be a biocompatible polymer such as various medically suitable acrylics and other plastics known and utilized in the art. In still other embodiments, the biocompatible material may be silicone or a silicone containing composition. Additionally, in various embodiments, the finish of the device may be to the standard for ophthalmic devices and may not create irritation to surrounding tissue. Such devices may be prepared by any method known and utilized in the art. For example, in embodiments in which the shunt or portions thereof are made from a biocompatible polymer or silicone, conventional injection molding, transfer molding, or any such process may be used.

In certain embodiments, the shunt or portions of the shunt may be composed of a material that can be coated with one or more materials that prevents and/or retards the attachment of cells and/or proteins present in the suprachoroidal space or in the fluid being transported by the shunt to the shunt or portions of the shunt that are coated with the material. In other embodiments, the shunt or portion of the shunt may be coated with a material that encourages cellular attachment to its external surface thereby providing a means by which the shunt may be held in place after implantation. In still other embodiments, one or more portions of the shunt may be coated with a material that encourages cellular attachment and other portions of the shunt may be coated with a material that discourages cellular attachment.

Various embodiments of the invention also include a shunt having one or more therapeutic agents incorporated into or coated onto the shunt or portions of the shunt. For example, in some embodiments, one or more therapeutic agents may be coated on an outer surface of the shunt or a portion of the outer surface of the shunt, and in other embodiments, one or more therapeutic agents may be coated on an inner surface of the shunt. In still other embodiments, the one or more therapeutic agents may be coated onto both an outer surface of the shunt or a portion thereof and an inner surface of the shunt or a portion thereof. Embodiments of the invention are not limited by the surfaces or portions of surfaces that may be coated. For example, in some embodiments, inner or outer surfaces of the elongate body, inner or outer surfaces of the insertion head, inner or outer surfaces of the connector, inner or outer surfaces of the plate, or combinations thereof may be coated. In other embodiments, therapeutic agents may be contained within the conduit, first branch of the conduit, second branch of the conduit or combinations thereof, and in still other embodiments, therapeutic agents may be contained within the conduit, first branch of the conduit, second branch of the conduit or combinations thereof and may coat inner or outer surfaces of the elongate body, inner or outer surfaces of the insertion head, inner or outer surfaces of the connector, inner or outer surfaces of the plate, or combinations thereof.

Embodiments of the invention are also not limited by the type of therapeutic agent or agents incorporated into the shunt. Non-limiting examples of therapeutic agents include agents for reduction of intraocular pressure, agents for prevention of fibrosis surrounding the inserted glaucoma drainage device, anti-inflammatory agents, immunosuppressive agents, and anti-proliferate agents, and combinations thereof. In certain embodiments, such therapeutic agents may include, for example, steroids, beta-blockers, alpha adrenergic agonists, alpha-2 antagonists, prostaglandin analogs, carbonic anhydride inhibitors, cholinesterase inhibitors, anti-fibrotic agents, antimicrobial agents, anti-inflammatory agents, antibiotics, and combinations thereof.

In various embodiments, the one or more therapeutic agents incorporated into or coated onto the shunt may be released locally from the shunt upon implantation, and in some embodiments, the one or more therapeutic agents may be released at a controlled rate and controlled amount following implantation. For example, in particular embodiments, the one or more therapeutic agents may be compounded or mixed with a release agent that reduces the rate of release of the therapeutic agent or allows the therapeutic agent to be time-released. In other embodiments, where more than one therapeutic agent is included in the shunt, each therapeutic agent may independently include a release agent, such that various therapeutic agents may be released at different times, or in a predetermined sequence.

Embodiments of the invention further include a surgical method for implanting any of the shunt embodied and described herein above into an eye. In some embodiments a first incision or slit may be made through the conjunctiva and the sclera at a location posterior to the limbus, the region of the sclera where the opaque white sclera begins to become clear cornea. For example, in certain embodiments, the first incision may be made from about 2 mm to about 9 mm or about 3 mm to about 6 mm posterior to the limbus, and in some embodiments, the first incision may be about 3 mm posterior to the limbus. In such embodiments, the first incision may be about the same width of the shunt or slightly larger than the width of the shunt. For example, in some embodiments, the width of the incision may be about 4 mm to about 7 mm, and in other embodiments, the incision may be about 5 mm to about 6 mm wide. In particular embodiments, a conventional cyclodialysis spatula may be inserted through the first incision into the supraciliary space to confirm correct anatomic position. After the first incision has been made, a portion of the shunt proximate to the back end of the body may be grasped by the surgical tool such as, for example, a forceps, and the forward end of the shunt may be oriented such that the longitudinal axis of the shunt is substantially co-axial to the longitudinal axis of the grasping end of the surgical tool. The shunt may be inserted into the tissue of the eye through the first incision into the supraciliary space. The shearing edge of the shunt may then be advanced anteriorly in the supraciliary space and inserted into and through the anterior chamber of the eye, and the shunt may be advanced anteriorly until a portion of the insertion head and the first end of the conduit is disposed within the anterior chamber of the eye. More particularly, the shearing edge of the insertion head may pass between the scleral spur and the ciliary body posterior to the trabecular meshwork and into the anterior chamber of the eye. As such, the first end of the conduit may be placed into fluid communication with the anterior chamber of the eye.

In embodiments in which the shunt includes a shoulder surface at the forward end of the elongate, the shoulder may be seated proximate an interior surface of the supraciliary space such that the shoulder surface and thus the elongate body are not introduced into the anterior chamber. Additionally, the shoulder surface may be positioned to aid in forming a tight seal at the incision into the anterior chamber to prevent leakage of fluid around the device and prevent unwanted anterior movement of the shunt following implantation.

The back end of the elongate body may be inserted into the suprachoroidal space of the eye such that the second end of the conduit may be placed into fluid communication with the suprachoroidal space. In such embodiments, the back end of the elongate body may be positioned under the posterior margin/lip of the scleral incision site to mitigate the risk of obstruction due to fibrosis or other tissue reactions associated with surgical wound healing that may otherwise result in the blockage of outflow through the second end of the conduit. The placement of the back end of the elongate body several millimeters posterior to the surgical incision may be done in a manner that is atraumatic to the sclera and choroid that border the suprachoroidal space.

The connector may be placed within the incision and the incision may be closed such that the connector protrudes through the incision and provides a passageway through the conjunctiva and the sclera and into the conjunctival space. For example, in some embodiments, a suture may be applied on one or both sides of the connector. In such embodiments, a suture which joins the opposing sides of the incision may be placed near the connector on at least one side of the incision, and such a suture may be sufficient to secure the shunt in place. In other embodiments, a suture may be placed on either side of the connector sufficiently near the connector to hold the shunt in place. In certain embodiments, closure of the incision may be effected by a means other than a suture. For example, in some embodiments, an adhesive may be used to close the incision.

In particular embodiments, an additional suture may be applied to the connector which encircles the connector and is positioned to obstruct flow of fluid through the connector by constricting the lumen of the connector by tightening the suture and allowing the lumen of the connector to dilate. In such embodiments, the additional suture may be applied during the implantation procedure to stop, for example, extraneous or excessive flow of fluid through the third end of the conduit, or the additional suture may be pre-applied such that the shunt is implanted with the additional suture in place. The additional suture may be removed at any time during the implantation procedure or during treatment following implantation and may provide a means for controlling flow of fluid from the anterior chamber by allowing out flow to increase if fluid pressure is not sufficiently decreased by flow through the second end of the conduit, or the additional suture may be removed in response during treatment to increase outflow of fluid in the event that the first branch of the conduit becomes blocked. In various embodiments of the method, any number of additional sutures may be applied to the incision in order to provide a proper closure in which fluid from the suprachoroidal space does not leak through the incision.

In other embodiments, one or more additional anchor sutures may be placed which secure the shunt within the suprachoroidal space, and in particular embodiments, the one or more additional anchor sutures may be anterior to the surgical incision. To facilitate fixation, the shunt 1, as depicted in FIG. 15 may have one or more spaced bore or suture hole 72 that extends between the upper 13 and lower 17 surfaces of the body 10 through which a suture can be passed to secure the shunt to the sclera. To simplify the surgical procedure, at least one suture may be preloaded into the bores of the device prior to inserting the device into the eye. However, it should be noted that multiple bore arrangements may be used for suturing the device to provide multiple possible locations for suturing the device dependant on the application, providing additional flexibility.

A plate may be placed in position over the connector. Positioning of the plate may be carried out by any means. For example, in some embodiments, the plate may be made from a flexible material that may be lifted while the incision is closed and then be repositioned against the sclera following closure. In other embodiments, the plate may be separated from the elongate body and connector and may be placed over the connector and connected to the connector by, for example, a compression snap or compression snaps, adhesive and the like. In particular embodiments, the plate may cover at least a portion of the incision when it is positioned against the sclera, and in some embodiments, the plate may cover the entire incision.

Upon implantation, the shunt forms a cyclodialysis with the conduit providing transverse communication of aqueous humor through the shunt along its length form the anterior chamber of the eye to the suprachoroidal space where the aqueous humor can be absorbed, and therefore, a reduction in pressure within the eye may result.

Another embodiment is directed to an ophthalmic shunt assembly including a shunt such as the shunt described herein above and an obturator. In such embodiments, an obturator or “stylet” may be removeably positioned within at least a portion of the interior of the conduit 40 thereby filling that portion of interior volume of the conduit 40 to prevent the conduit 40 from becoming obstructed as the shunt is advanced into place. For example, in some embodiments, the obturator may be positioned to fill the entire conduit, such that both the first end 41 of the conduit 40 and the opening at the back end 42 of the conduit 40 are completely filled by the obturator. In some such embodiments, the obturator may be flush with the opening of the first end 42 of the conduit 40, or in other embodiments, the obturator may extend beyond and protrude from the first end 42 of the conduit 40. Therefore, the obturator may be configured to block the first end 42 of the conduit 40 and may prevent accumulation of tissue and blockage of the conduit 40 that could otherwise be forced into the first end 42 of the conduit 40 as the insertion head 20 is forcefully pressed though the eye tissue.

In a further aspect, the obturator may provide a means for “priming” the conduit. In such embodiments, fluid may displace air or the material of the obturator as it is removed from the conduit.

In another aspect, the obturator may be configured to act as the insertion instrument itself and obviate the need to grasp the device on its outside surfaces or surface features. For example, an exemplary embodiment of the obturator may include a handle portion. The handle portion of some embodiments may be integral with the obturator such that the handle is formed from the same material as the obturator. In such embodiments, the obturator may make up a mount portion of the device. In other embodiments, the handle portion may be removably attached to the obturator. The handle portion may have a proximal end portion and a distal end portion. The distal end portion may be ergonomically designed to orient the hand of the surgeon, upon his or her employment of the obturator in a naturally functional position. The proximal end portion may be designed to facilitate proper placement of shunt. For example, the proximal end may be angled or curved such that the shunt is properly or conveniently aligned when the operator grasps the distal end portion. In one embodiment, the proximal end portion may extend along a longitudinal axis, and the distal end portion is oriented relative to the longitudinal axis of the proximal end portion at an angle, for example, between 90 and 150 degrees. However, it will be appreciated that angles outside of this range may be necessary, and may be employed by one skilled in the art which may or may not maintain the ergonomic character of the handle. Further, the union of the proximal end portion and distal end portion is preferably rounded and or smooth to avoid sharp edges which could cause injury to surrounding tissues upon insertion of the shunt. In some embodiments, the obturator may be prepared from the same material as the shunt, and in other embodiments, the obturator may be prepared from a different material than the shunt.

The obturator may be configured to create a temporary, selectively releasable, engagement with the means for mounting provided by the elongate body of the shunt. In one aspect, to achieve the desired engagement, the obturator may have a first end and a second end, wherein the first end may be connected to the distal end portion of the handle, and extends outwardly toward to the second end. At least a portion of the second end may be configured for operative receipt by the conduit in the shunt, such that the shunt may be selectively fixed to the second end of the obturator, which ensures that movement of the second end of the obturator may cause the same relative movement of the mounted shunt. In certain embodiments, the first end may be flush with the distal end of the conduit thereby blocking the distal opening of the conduit.

In some embodiments, at least a portion of the mount portion may be selectively withdrawn within a portion of the distal end portion of the handle. It is further contemplated that the distal end portion of the handle can define a stop that may be configured to prevent the rearward movement of the shunt as the mount portion is withdrawn from the distal end portion of the handle.

In some embodiments, at least a portion of the second end of the obturator has a shape that closely conforms to a portion of the interior of the conduit. For example, in one embodiment, the conduit may have a wedge shape such that the width of the conduit decreases from back to front. Complementarily, at least a portion of the mount portion of the obturator has a wedge shape such that the width of the mount portion accordingly decreases moving longitudinally from the first end to the second end.

In another embodiment, the second end of the mounting portion can be configured to effectively block the first end of the conduit. In this aspect, the obturator forms a shoulder surface that is configured to operatively engage the back end of the body of the shunt. This allows a pushing force to be applied to the back end of the shunt. In another embodiment, the obturator may define a plurality of tabs that are connected to edge portions of the shoulder surface and that extend outwardly away from the shoulder surface. In this example, a plurality of tabs may define a notch that is configured to make releasable contact portions of the exterior surface of the shunt proximate the back end of the shunt. This would allow for control over the orientation of the shunt as it is mounted onto the obturator and would insure that movement of the second end of the obturator causes the same relative movement of the mounted shunt.

In one aspect, the first and second prongs of the obturator and the slots of the shunt may be configured such that upon insertion of the prongs into the slots, the shunt is positionally fixed with respect to the obturator. Thus, the shunt may be readily implantable as it resists twisting relative to and about the mounting portion of the obturator. In this aspect, the first and second prongs add additional support to the connection between the mount portion of the obturator and the shunt to decrease slippage and allow for more precise control of the shunt during implantation. It will be noted, however, that additional or fewer prongs may be utilized as the situation requires, and that the inclusion of an embodiment having a plurality of prongs is merely for illustrative purposes and is not meant to be limiting. Further, substitute prong cross-sectional geometric shapes, such as half circle, triangular, and the like are also contemplated.

Additional prongs may be formed in the mount portion of the obturator that may be configured to be operatively received into the conduit. In this aspect, the additional prong performs substantially the same function as the prong in the single pronged embodiment that is described above.

The surgical method for implanting the device of the present invention into an eye will be explained. A first incision or slit is made through the conjunctiva and the sclera at a location rearward of the limbus, that is, posterior to the region of the sclera at which the opaque white sclera starts to become clear cornea. Preferably, the first incision may be made about 2 mm to about 9 mm or about 3 mm to about 6 mm or about 3 mm posterior to the limbus. Also, the first incision is made slightly larger than the width of the implant device. A conventional cyclodialysis spatula may be inserted through the first incision into the supraciliary space to confirm correct anatomic position.

The obturator may be inserted into the shunt so that the shunt is oriented properly. As discussed above, the obturator may penetrate the conduit, or include additional prongs for holding the shunt in position. By manipulation of the obturator, the shunt may be disposed through the first incision and into the supraciliary space of the eye. The shearing edge of the shunt may then be advanced anteriorly in the supraciliary space and may be inserted into and through the anterior chamber angle of the eye. More particularly, the shearing edge of the insertion head may pass between the scleral spur and the ciliary body posterior to the trabecular meshwork. The shunt may be advanced anteriorly until a portion of the insertion head and the first end of the conduit is disposed within the anterior chamber of the eye. The tissue surrounding the incision can be stretched about the exterior of the insertion head to substantially form a fluid seal or water-tight seal about the insertion head (at the junction between the suprachoroidal space and the anterior chamber). Thus, the first end of the conduit is placed into fluid communication with the anterior chamber of the eye. Following removal of the obturator, the back end of the elongate body may be disposed into the suprachoroidal space of the eye so that the second end of the conduit is placed into fluid communication with the suprachoroidal space.

In one aspect, the obturator may allow for a less traumatic shunt introduction and placement than other available surgical methods. In one exemplified aspect, the obturator may preclude obstruction of the conduit. As shown in the figures, the obturator may be removably positioned within at least a portion of the conduit, thereby filing at least a portion of the interior volume of the conduit proximate the first end of the conduit and preventing obstruction of the first end of the conduit. Thus, in one aspect, the obturator can be configured to selectively block the first end of the conduit to prevent any accumulation of tissue that could cause partial or full blockage of the conduit. Once the shunt is installed, removal of the obturator from the conduit may result in an aspiration of fluid into the conduit, thereby establishing a fluid flow through the conduit from the anterior chamber into the suprachoroidal space.

In another aspect, it is contemplated that the second end of the obturator can be configured to extend outwardly beyond the exterior surface of the insertion head. In this aspect, at least a portion of the second end of the obturator can define a shearing edge that is configured for penetrating tissue. In this aspect, the shearing edge can be used as a dilator or instrument for dissection.

The shunt may then be sutured to a portion of the sclera to aid in fixating the shunt. The first incision is subsequently sutured closed. As one will appreciate, the suture used to fix the shunt may also be used to close the first incision. In a further aspect, the conduit of the shunt may be primed by withdrawing the obturator from the conduit, which aspirates fluid into the conduit while displacing the material of the obturator.

What is claimed is:

1. An ophthalmic shunt, comprising:
an elongate body having a forward end, a spaced back end, an upper surface, and a lower surface;
an insertion head extending from the forward end of the elongate body and being continuous with the elongate body, the insertion head defining a shearing edge constructed and arranged for cutting eye tissue;
a conduit having a first end defined on the insertion head and a first branch extending through the elongate body from the forward end to the back end of the elongate body and a second branch extending through the elongate body to the upper surface of the elongate body;
a connector extending from the upper surface of the elongate body, the connector encompassing and lengthening the second branch, wherein the second branch forms a lumen within the connector;
a plate having an upper and a lower surface, the lower surface of the plate extending from the connector opposite the elongate body and the connector creating a space between the upper surface of the elongate body and the lower surface of the plate.

2. The shunt of claim 1, wherein the elongate body is configured to position at least a portion of the insertion head and the first end of the conduit through an incision formed by the shearing edge of the insertion head and capable of being in fluid communication with the anterior chamber of the eye.

3. The shunt of claim 1, wherein the elongate body has an arcuate shape along at least a portion of its length that is adapted to extend along the curvature of the sclera.

4. The shunt of claim 1, wherein the elongate body has a substantially fusiform cross-sectional shape.

5. The shunt of claim 1, wherein the plate extends beyond at least one edge of the elongate body.

6. The shunt of claim 1, wherein the plate has a shape selected from the group consisting of polygonal, rounded polygonal, circular, oval, and elliptical.

7. The shunt of claim 1, wherein the plate is contoured to cover at least a portion of the sclera.

8. The shunt of claim 1, wherein at least one axis of the plate has a diameter or width of greater than about 2 mm.

9. The shunt of claim 1, wherein at least one axis of the plate has a diameter or width of from about 3 mm to about 9 mm.

10. The shunt of claim 1, wherein at least one axis of the plate has a diameter or width of about 6 mm.

11. The shunt of claim 1, wherein the upper surface of the plate is convex.

12. The shunt of claim 11, wherein the convex upper surface of the plate has a curvature that is substantially similar to adjacent sclera when the shunt is implanted.

13. The shunt of claim 1, wherein the upper surface of the elongate body and the lower surface of the plate are arranged and spaced to receive a sclera of an eye.

14. The shunt of claim 1, wherein the lower surface of the plate is substantially flat.

15. The shunt of claim 1, wherein the lower surface of the plate is concave.

16. The shunt of claim 15, wherein the concave lower surface of the plate has a curvature that is steeper than adjacent sclera when the shunt is implanted such that a convex space is created between the sclera and the lower surface of the plate when the shunt is implanted.

17. The shunt of claim 1, wherein at least a portion of the lower surface of the plate is textured.

18. The shunt of claim 17, wherein the texture of the textured lower surface is selected from the group consisting of corrugations, fingers, bumps, concentric circles, portions of concentric circles, and combinations thereof.

19. The shunt of claim 1, wherein the upper surface of the plate is substantially co-planar with the lower surface of the elongate body.

20. The shunt of claim 1, wherein the upper surface of the plate is convex and the lower surface of plate is concave.

21. The shunt of claim 20, wherein the curvature of the convex upper surface and the curvature of the concave lower surface is substantially the same.

22. The shunt of claim 1, wherein the connector has a height of from about 0.5 mm to about 0.8 mm from the upper surface of the elongate body to the lower surface of the plate.

23. The shunt of claim 1, wherein the connector has a height of about 0.6 mm from the upper surface of the elongate body to the lower surface of the plate.

24. The shunt of claim 1, wherein a joint between the connector and the plate is at a midpoint of the plate.

25. The shunt of claim 1, wherein a joint between the connector and the plate is offset from a midpoint of the plate.

26. The shunt of claim 25, wherein the joint between the connector and the plate is offset toward an anterior portion of the plate.

27. The shunt of claim 25, wherein the joint between the connector and the plate is positioned such than an outer surface of the connector and an outer edge of the plate are separated by about 2 mm or more.

28. The shunt of claim 27, wherein the outer edge of the plate is defined on an anterior portion of the plate.

29. The shunt of claim 1, wherein the connector is separated from the back end of the elongate body by about 1 mm or more.

30. The shunt of claim 1, wherein at least a portion of the lumen of the connector comprises a flow regulator.

31. The shunt of claim 30, wherein the flow regulator is selected from the group consisting of a valve, a membrane, a porous material, a flap, and combinations thereof.

32. The shunt of claim 30, wherein the flow regulator is comprised of a biodegradable material, a non-biodegradable material, or a combination thereof.

33. The shunt of claim 1, wherein a second end of the second branch is defined on the upper surface of the plate.

34. The shunt of claim 33, wherein the upper surface of the plate further comprises a flow regulator position to regulate the flow of liquid through the second end of the conduit.

35. The shunt of claim 33, wherein the flow regulator is selected from the group consisting of a valve, a membrane, a porous material, a flap, and combinations thereof.

36. The shunt of claim 33, wherein the flow regulator is comprised of a biodegradable material, a non-biodegradable material, or a combination thereof.

37. The shunt of claim 1, wherein a second end of the second branch is defined on a portion of the connector.

38. The shunt of claim 37, wherein the second end of the second branch is positioned below the lower surface of the plate.

39. The shunt of claim 37, wherein the second end of the second branch comprises one or more openings in the connector perpendicular to the lumen.

40. The shunt of claim 37, wherein the upper surface of the plate is continuous such that no opening for the second branch is defined on the upper surface of the plate.

41. The shunt of claim 37, further comprising an aperture to the second branch defined on an upper surface of the plate.

42. The shunt of claim 41, wherein the aperture further comprises a flow regulator positioned to regulate the flow of liquid through the aperture.

43. The shunt of claim 42, wherein the flow regulator is selected from the group consisting of a valve, a membrane, a porous material, a flap, and combinations thereof.

44. The shunt of claim 43, wherein the membrane or the porous material can be at least partially removed by laser.

45. The shunt of claim 1, wherein the shunt is comprised of a material selected from the group consisting of biocompatible metals, gold, platinum, nickel, molybdenum, titanium, biocompatible metal alloys, biocompatible polymers, silicone, and combinations thereof.

46. The shunt of claim 45, wherein the elongate body comprises a material selected from the group consisting of rigid materials and semi-rigid materials.

47. The shunt of claim 1, wherein the plate comprises a flexible material.

48. The shunt of claim 47, wherein the plate is comprised of silicone.

49. The shunt of claim 47, wherein the plate is comprised of a flexible biocompatible polymer.

50. The shunt of claim 1, wherein the connector comprises a material selected from the group consisting of a flexible material, a semi-rigid material, a rigid material, and combinations thereof.

51. The shunt of claim 1, wherein the connector further comprises a suture encircling the connector and positioned and arranged to obstruct flow of fluid through the connector.

52. The shunt of claim 51, wherein the suture is selected from the group consisting of releasable sutures, biodegradable sutures, and combinations thereof.

53. The shunt of claim 1, further comprising one or more therapeutic agent.

54. The shunt of claim 53, where the therapeutic agent is selected from the group consisting of steroids, beta blockers, alpha-2 antagonists, carbonic anhydride inhibitors, prostaglandin analogues, anti-fibrotic agents, anti-inflammatory agents, antimicrobial agents, and combinations thereof.

55. The shunt of claim 53, wherein the one or more therapeutic agents are contained within the conduit, the first branch, the second branch, or combinations thereof.

56. The shunt of claim 53, wherein the one or more therapeutic agents are coated on outer or inner surfaces of the elongate body, coated on outer or inner surfaces of the insertion head, coated on outer or inner surfaces of the connector, coated on outer or inner surfaces of the plate, or combinations thereof.

57. A method for treating glaucoma in an eye comprising:
inserting at least a portion of a first end of a biocompatible ophthalmic shunt through the sclera and suprachoroidal space into the anterior chamber of an eye such that at least a portion of the first end is in fluid communication with the anterior chamber of the eye;
positioning a second portion of the shunt into a suprachoroidal space of the eye such that at least a portion the second portion of the shunt is in fluid communication with the suprachoroidal space; and
positioning a third portion of the shunt into the subconjunctival space of the eye such that at least a portion of the third portion of the shunt is in communication with the subconjunctival space.

58. The method of claim 57, wherein the first end, the second portion, and the third portion are connected by a branched conduit.

59. The method of claim 57, wherein flow of fluid through the third portion of the shunt is at least partially obstructed when the third portion is initially positioned.

60. The method of claim 57, further comprising removing the obstruction when flow of fluid through the second portion becomes blocked.

61. The method of claim 57, further comprising removing the obstruction when pressure within the anterior chamber of the eye is insufficiently reduced to effect treatment.

62. The method of claim 57, further comprising applying a suture to the third portion of the shunt to obstruct flow of fluid through the third portion of the shunt.

63. The method of claim 62, wherein the suture is selected from the group consisting of releasable sutures and biodegradable sutures.

64. The method of claim 62, further comprising releasing the suture when flow of fluid through the second portion becomes blocked.

65. The method of claim 62, further comprising releasing the suture when pressure within the anterior chamber of the eye is insufficiently reduced to effect treatment.

66. The method of claim 57, wherein the shunt comprises a flow regulator selected from the group consisting of a membrane, a porous material, and combinations thereof, and wherein the method further comprises removing at least a portion of the membrane, porous material, or a combination thereof when flow of fluid through the second portion is insufficient to reduce to effect treatment.

67. The method of claim 66, wherein removing at least a portion of the membrane, porous material, or a combination thereof comprises applying a laser to the membrane, porous material or combination thereof.

68. The method of claim 57, wherein the shunt comprises a flow regulator selected from the group consisting of a valve, a flap or a combination thereof, and wherein the method further comprises opening the valve, flap, and combinations thereof when flow of fluid through the second portion is insufficient to effect treatment.
